(19) United States
(12) Patent Application Publication
(10) Pub. No.: US 2013/0171178 A1
(43) Pub. Date: Jul. 4, 2013

(54) Immunogenic Peptides of Xage-1

(71) Applicants: Jay A. Berzofsky, Bethesda, MD (US); Ira H. Pastan, Potomac, MD (US); Masaki Terabe, Bethesda, MD (US)

(72) Inventors: Jay A. Berzofsky, Bethesda, MD (US); Ira H. Pastan, Potomac, MD (US); Masaki Terabe, Bethesda, MD (US)

(73) Assignee: THE GOVERNMENT OF THE UNITED STATES AS REPRESENTED BY THE SECRETARY OF HEALTH AND HUMAN SERVICES, Rockville, MD (US)

(21) Appl. No.: 10/582,703

(22) PCT Filed: Dec. 13, 2004
(86) PCT No.: PCT/US2004/041639

§ 371 Date: May 15, 2007

Related U.S. Application Data
(60) Provisional application No. 60/529,025, filed on Dec. 12, 2003.

(51) Int. Cl. C07K 7/06; A61K 39/00; A61K 45/06; C07K 7/08; C12P 21/06; C07H 21/04; C07K 14/82

(52) U.S. Cl. CPC C07K 7/06; A61K 39/0011; A61K 45/06
USPC 424/185.1; 530/328; 530/327; 530/329; 435/69.1; 435/320.1; 435/325; 536/23.5

(57) ABSTRACT

XAGE-1 is a gene expressed in a number of important human cancers, including prostate cancer, lung cancer, breast cancer, ovarian cancer, glioblastoma, pancreatic cancer, and melanoma. It has now been discovered that peptides of fifty or fewer amino acids comprising the sequence X₁X₂X₃PSAPSPX₄ (SEQ ID NO:5), where X₁ is any amino acid and is preferably G or Y; X₂ is selected from the group consisting of L, M, A, I, V, and T, with L and M being preferred; X₃ is a hydrophobic residue, M or A; and X₄ is V, M, L, A, I, or T, and is preferably V, bind to the HLA-A2 MHC class I molecule, and can be used to raise immune responses to XAGE-1-expressing cancers. In some embodiments, the P at position 7, the S at position 8, or the P at position 9, can be omitted to create a 9 amino acid peptide. The invention provides immunogenic peptides, nucleic acids encoding them, vectors comprising the nucleic acids, uses of the peptides and nucleic acids for manufacture of medicaments, methods of using the peptides and nucleic acids, and compositions of the peptides or nucleic acids in pharmaceutically acceptable carriers.

### CROSS-REFERENCES TO RELATED APPLICATIONS

This application claims the benefit of U.S. Provisional Application No. 60/529,025, filed Dec. 12, 2004, the contents of which are hereby incorporated by reference.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK.

NOT APPLICABLE

### BACKGROUND OF THE INVENTION

Cancer-testis (CT) antigens are a distinct class of differentiation, antigens that have a restricted pattern of expression in normal tissues (De Smet, C. et al., Eye., 11:243-248 (1997); Chen, Y. T. Cancer J. Sci. Am., 5:16-17 (1999); Gillespie, A. et al., Br. J. Cancer., 78:816-821 (1998)). Some thoroughly studied CT antigens are MAGE, BAGE, GAGE and LAGE/NY-ESO-1 (Chen, Y. T. Cancer J. Sci. Am., 5:16-17 (1999); Gillespie, A. et al., Br. J. Cancer., 78:816-821 (1998); Lucas, S. et al., Cancer Res., 58:743-752 (1998); Jungbluth, A. A. et al., Int. J. Cancer., 85:460-465 (2001); Chen, Y. T. et al., Proc. Natl. Acad. Sci. U S A., 95:6919-6923 (1998); Boel, P. et al., Immunity., 2:167-175 (1995); Backer, O. et al., Cancer Res., 59:3157-3165 (1991); De Plaen, E. et al., Immunogenetics. 40:360-369 (1994); Chen, Y. T. et al., Cell Genet., 79:237-240 (1997)). These genes are primarily expressed in the primitive germ cells, spermatogonia, and in the normal testis. Malignant transformation is often associated with activation or derepression of silent CT genes, and this results in the expression of CT antigens in a variable proportion of a wide range of human tumors. Recently, several additional members were added to the CT antigen family. These include various PAGEs, PRAME, SSX, SCP-1, C17 and MAGEC1 and MAGED1 (Brinkman, U. et al., Proc. Natl. Acad. Sci. USA., 95:10757-10762 (1998); Lucas, S. et al., Cancer Res., 58:743-752 (1998); Gure, A. O. et al., Int. J. Cancer., 85:726-732 (2000); Tureci, O. et al., Int. J. Cancer., 77:19-23 (1998); Tureci, O. et al., Proc. Natl. Acad. Sci. USA., 95:5211-5216 (1998); Pold, M. et al., Genomics., 59:161-167 (1999); Watari, K. et al., FEBS Lett., 466: 367-371 (2000)). Identification of new CT antigens or new family members continues to be pursued in the cancer research field.

Three related genes, termed XAGEs, were identified by homology walking using the dbEST database (Brinkmann, U. et al., Cancer Res., 59:1445-1448 (1999) (“Brinkmann 1999”)). ESTs of the XAGE group were found in various cDNA libraries. The XAGE-1 cluster contained ESTs from testis, germ cell tumors, and from some relatively rare tumors of bone and muscle most frequently found in children: Ewing's sarcoma, and alveolar rhabdomyosarcoma. The authors of Brinkmann 1999 reported, however, that there appeared to be two reading frames, and that the second did not contain a start codon until about halfway through the sequence. Due to the uncertainty with translation, the authors were unable to report a protein encoded by the gene they named “XAGE-1.”

International Publication No. WO 02/18584 stated that XAGE-1 was translated as two proteins, a 9 kD protein termed “p9” and a 16.3 kD protein termed “p16.” The sequences of both proteins were set forth in the WO publication. The WO publication stated that XAGE-1 was found to be expressed in a number of important human cancers, including prostate cancer, lung cancer, breast cancer, ovarian cancer, glioblastoma, pancreatic cancer, T cell lymphoma, melanoma, and histocytic lymphoma, and that the two proteins, or immunogenic fragments or analogs of them, could be administered to persons with such cancers to raise an immune response. See also, Liu et al., Cancer Res. 60:4752-55 (2000). Given the need to increase immunogenic responses to XAGE-expressing cancers, it would be desirable to identify specific peptides that would raise an immune response to such cancers.

### BRIEF SUMMARY OF THE INVENTION

In a first group of embodiments, the invention provides isolated immunogenic peptides of 50 or fewer amino acids comprising an amino acid sequence X₁X₂X₃PSAPSPX₄ (SEQ ID NO:5), wherein X₁ can be any amino acid; X₂ can be L, M, A, I, V, or T; X₃ can be a hydrophobic residue, methionine or alanine; and X₄ can be V, M, L, A, I, or T. In some embodiments, X₁ is tyrosine (SEQ ID NO:34), in some, X₂ is leucine (SEQ ID NO:35), in some X₃ is methionine (SEQ ID NO:36), and in some, X₄ is valine (SEQ ID NO:37). In some embodiments, the peptide comprises an amino acid sequence selected from the group consisting of GVFPSAPSPV (SEQ ID NO:6), YVFPSAPSPV (SEQ ID NO:7), GLFPSAPSPV (SEQ ID NO:8), GVMPSAPSPV (SEQ ID NO:9), YLFPSAPSPV (SEQ ID NO:10), and GLMPSAPSPV (SEQ ID NO:11). In some embodiments, the peptide is a ten amino acid peptide having an amino acid sequence selected from the group consisting of GVFPSAPSPV (SEQ ID NO:6), YVFPSAPSPV (SEQ ID NO:7), GLFPSAPSPV (SEQ ID NO:8), GVMPSAPSPV (SEQ ID NO:9), YLFPSAPSPV (SEQ ID NO:10), and GLMPSAPSPV (SEQ ID NO:11).

In another group of embodiments, the invention provides compositions comprising an isolated immunogenic peptide of 50 or fewer amino acids comprising an amino acid sequence X₁X₂X₃PSAPSPX₄ (SEQ ID NO:5), wherein X₁ can be any amino acid; X₂ can be L, M, A, I, V, or T; X₃ can be a hydrophobic residue, methionine or alanine; and X₄ can be V, M, L, A, I, or T, and a pharmaceutically acceptable carrier. In some embodiments, X₁ is tyrosine (SEQ ID NO:34), in some, X₂ is leucine (SEQ ID NO:35), in some X₃ is methionine (SEQ ID NO:36), and in some, X₄ is valine (SEQ ID NO:37). In some embodiments, the peptide comprises an amino acid sequence selected from the group consisting of GVFPSAPSPV (SEQ ID NO:6), YVFPSAPSPV (SEQ ID NO:7), GLFPSAPSPV (SEQ ID NO:8), GVMPSAPSPV (SEQ ID NO:9), YLFPSAPSPV (SEQ ID NO:10), and GLMPSAPSPV (SEQ ID NO:11). In some embodiments, the peptide is a ten amino acid peptide having an amino acid sequence selected from the group consisting of GVFPSAPSPV (SEQ ID NO:6), YVFPSAPSPV (SEQ ID NO:7), GLFPSAPSPV (SEQ ID NO:8), GVMPSAPSPV (SEQ ID NO:9), YLFPSAPSPV (SEQ ID NO:10), and GLMPSAPSPV (SEQ ID NO:11).

In yet an additional group of embodiments, the invention provides for the use of an isolated immunogenic peptide of fifty or fewer amino acids comprising a sequence of X₁X₂X₃PSAPSPX₄ (SEQ ID NO:5), wherein X₁ can be any amino acid; X₂ can be L, M, A, I, V, or T; X₃ can be a hydrophobic residue, methionine or alanine; and X₄ can be V, M, L, A, I, or T, for the manufacture of a medicament to raise an immune response to cells expressing a protein encoded by XAGE-1. In some embodiments, X₁ is tyrosine (SEQ ID NO:34), in some X₂ is a leucine (SEQ ID NO:35), and in some X₃ is a methionine (SEQ ID NO:36). In some uses, the peptide comprises an amino acid sequence selected from the group consisting of GVFPSAPSPV (SEQ ID NO:6), YVFPSAPSPV (SEQ ID NO:7), GLFPSAPSPV (SEQ ID NO:8), GVMPSAPSPV (SEQ ID NO:9), YLFPSAPSPV (SEQ ID NO:10), and GLMPSAPSPV (SEQ ID NO:11). In some embodiments, the peptide is a ten amino acid peptide having a sequence selected from the group consisting of GVFPSAPSPV (SEQ ID NO:6), YVFPSAPSPV (SEQ ID NO:7), GLFPSAPSPV (SEQ ID NO:8), GVMPSAPSPV (SEQ ID NO:9), YLFPSAPSPV (SEQ ID NO:10), and GLMPSAPSPV (SEQ ID NO:11).

In still another group of embodiments, the invention provides methods of inhibiting the growth of an XAGE-1-expressing cancer cell, comprising administering a peptide of fifty or fewer amino acids, said peptide comprising a sequence of X₁X₂X₃PSAPSPX₄ (SEQ ID NO:5), wherein X₁ can be any amino acid; X₂ can be L, M, A, I, V, or T; X₃ can be a hydrophobic residue, methionine or alanine; and X₄ can be V, M, L, A, I, or T, wherein administration of the peptide stimulates or activates cytotoxic T lymphocytes, thereby inhibiting growth of said XAGE-1-expressing cancer cell. In some embodiments, X₁ is a tyrosine (SEQ ID NO:34), in some X₂ is a leucine (SEQ ID NO:35), and in some X₃ is a methionine (SEQ ID NO:36). In some embodiments, the peptide comprises an amino acid sequence selected from the group consisting of GVFPSAPSPV (SEQ ID NO:6), YVFPSAPSPV (SEQ ID NO:7), GLFPSAPSPV (SEQ ID NO:8), GVMPSAPSPV (SEQ ID NO:9), YLFPSAPSPV (SEQ ID NO:10), and GLMPSAPSPV (SEQ ID NO:11). In some embodiments, the peptide has an amino acid sequence selected from the group consisting of GVFPSAPSPV (SEQ ID NO:6), YVFPSAPSPV (SEQ ID NO:7), GLFPSAPSPV (SEQ ID NO:8), GVMPSAPSPV (SEQ ID NO:9), YLFPSAPSPV (SEQ ID NO:10), and GLMPSAPSPV (SEQ ID NO:11). The methods can further comprise administering an immunostimulant or an antagonist of immunosuppressive cytokines.

In yet another group of embodiments, the invention provides isolated nucleic acids encoding a peptide of fifty or fewer amino acids comprising a sequence X₁X₂X₃PSAPSPX₄ (SEQ ID NO:5), wherein X₁ can be any amino acid; X₂ can be L, M, A, I, V, or T; X₃ can be a hydrophobic residue, methionine or alanine; and X₄ can be V, M, L, A, I, or T. In some embodiments, X₁ is tyrosine (SEQ ID NO:34). In some embodiments, X₂ is leucine (SEQ ID NO:35). In some embodiments, X₃ is methionine (SEQ ID NO:36). In some embodiments, the peptide comprises an amino acid sequence selected from the group consisting of GVFPSAPSPV (SEQ ID NO:6), YVFPSAPSPV (SEQ ID NO:7), GLFPSAPSPV (SEQ ID NO:8), GVMPSAPSPV (SEQ ID NO:9), YLFPSAPSPV (SEQ ID NO:10), and GLMPSAPSPV (SEQ ID NO:11). In some embodiments, the peptide has an amino acid sequence selected from the group consisting of GVFPSAPSPV (SEQ ID NO:6), YVFPSAPSPV (SEQ ID NO:7), GLFPSAPSPV (SEQ ID NO:8), GVMPSAPSPV (SEQ ID NO:9), YLFPSAPSPV (SEQ ID NO:10), and GLMPSAPSPV (SEQ ID NO:11).

In yet further embodiments, the invention provides vectors comprising an isolated nucleic acid as described in the preceding paragraph, operably linked to a promoter. In some embodiments, the nucleic acid sequence encodes a peptide comprising an amino acid sequence selected from the group consisting of GVFPSAPSPV (SEQ ID NO:6), YVFPSAPSPV (SEQ ID NO:7), GLFPSAPSPV (SEQ ID NO:8), GVMPSAPSPV (SEQ ID NO:9), YLFPSAPSPV (SEQ ID NO:10), and GLMPSAPSPV (SEQ ID NO:11).

In some embodiments, the invention provides a vector as described in the paragraph above and a pharmaceutically acceptable carrier. In some embodiments, the vector encodes a peptide comprising an amino acid sequence selected from the group consisting of GVFPSAPSPV (SEQ ID NO:6), YVFPSAPSPV (SEQ ID NO:7), GLFPSAPSPV (SEQ ID NO:8), GVMPSAPSPV (SEQ ID NO:9), YLFPSAPSPV (SEQ ID NO:10), and GLMPSAPSPV (SEQ ID NO:11).

In another group of embodiments, the invention provides uses of the nucleic acid and vectors described above for the manufacture of a medicament to inhibit the growth of a XAGE-1-expressing cancer cell in a subject. In some embodiments, the nucleic acid encodes a peptide comprising an amino acid sequence selected from the group consisting of GVFPSAPSPV (SEQ ID NO:6), YVFPSAPSPV (SEQ ID NO:7), GLFPSAPSPV (SEQ ID NO:8), GVMPSAPSPV (SEQ ID NO:9), YLFPSAPSPV (SEQ ID NO:10), and GLMPSAPSPV (SEQ ID NO:11).

In yet another group of embodiments, the invention provides methods of inhibiting the growth of an XAGE-1-expressing cancer cell comprising administering an isolated nucleic acid sequence encoding a peptide of fifty or fewer amino acids, said peptide comprising of the sequence X₁X₂X₃PSAPSPX₄ (SEQ ID NO:5), wherein: X₁ can be any amino acid; X₂ can be L, M, A, I, V, or T; X₃ can be a hydrophobic residue, methionine or alanine; and X₄ can be V, M, L, A, I, or T; wherein administration of the nucleic acid sequence results in expression of the peptide, which stimulates or activates cytotoxic T lymphocytes, thereby inhibiting the growth of said XAGE-1-expressing cancer cell in said mammal. In some embodiments, X₁ is tyrosine (SEQ ID NO:34), in some X₂ is leucine (SEQ ID NO:35), and in some, X₃ is methionine (SEQ ID NO:36). In some embodiments, the peptide comprises an amino acid sequence selected from the group consisting of GVFPSAPSPV (SEQ ID NO:6), YVFPSAPSPV (SEQ ID NO:7), GLFPSAPSPV (SEQ ID NO:8), GVMPSAPSPV (SEQ ID NO:9), YLFPSAPSPV (SEQ ID NO:10), and GLMPSAPSPV (SEQ ID NO:11). In other embodiments, the peptide has an amino acid sequence selected from the group consisting of GVFPSAPSPV (SEQ ID NO:6), YVFPSAPSPV (SEQ ID NO:7), GLFPSAPSPV (SEQ ID NO:8), GVMPSAPSPV (SEQ ID NO:9), YLFPSAPSPV (SEQ ID NO:10), and GLMPSAPSPV (SEQ ID NO:11).

In still another group of embodiments, the invention provides methods for stimulating or expanding T cells, or both, in vitro, comprising contacting T cells with a synthetic or recombinant amino acid sequence X₁X₂X₃PSAPSPX₄ (SEQ ID NO:5), wherein: X₁ can be any amino acid; X₂ can be L, M, A, I, V, or T; X₃ can be a hydrophobic residue, methionine or alanine; and X₄ can be V, M, L, A, I, or T; thereby stimulating or expanding said T cells, or both. In some embodiments, X₁ is tyrosine (SEQ ID NO:34), in some X₂ is leucine (SEQ ID NO:35), and in some X₃ is methionine (SEQ ID NO:36). In some embodiments, the peptide comprises an amino acid sequence selected from the group consisting of GVFPSAPSPV (SEQ ID NO:6), YVFPSAPSPV (SEQ ID NO:7), GLFPSAPSPV (SEQ ID NO:8), GVMPSAPSPV (SEQ ID NO:9), YLFPSAPSPV (SEQ ID NO:10), and GLMPSAPSPV (SEQ ID NO:11). In some embodiments, the peptide has an amino acid sequence selected from the group consisting of GVFPSAPSPV (SEQ ID NO:6), YVFPSAPSPV (SEQ ID NO:7), GLFPSAPSPV (SEQ ID NO:8), GVMPSAPSPV (SEQ ID NO:9), YLFPSAPSPV (SEQ ID NO:10), and GLMPSAPSPV (SEQ ID NO:11). The T cells can be isolated, for example, from a patient's bone marrow, or a fraction thereof, or from peripheral blood, or a fraction thereof. The T cells may be contacted with the peptide by contacting the T cells with an antigen presenting cell pulsed with, transduced to express, or differentiated from a cell transduced with a nucleic acid encoding, the peptide. In some embodiments, the T cells are contacted with an antigen presenting cell pulsed with, transduced to express, or differentiated from a cell transduced with a nucleic acid encoding, a peptide having an amino acid sequence selected from the group consisting of GVFPSAPSPV (SEQ ID NO:6), YVFPSAPSPV (SEQ ID NO:7), GLFPSAPSPV (SEQ ID NO:8), GVMPSAPSPV (SEQ ID NO:9), YLFPSAPSPV (SEQ ID NO:10), and GLMPSAPSPV (SEQ ID NO:11). In some embodiments, the T cells are CD8+ T cells.

In still another group of embodiments, the invention provides methods for stimulating or expanding T cells in vitro, comprising contacting said T cells with an isolated

### BRIEF DESCRIPTION OF THE DRAWINGS

FIG. 1. Diagram of the XAGE-1 transcripts. The complete nucleic acid sequence of XAGE-1 shown, with untranslated 5′ and 3′ ends, is SEQ ID NO:1. The polyadenylation signal is italicized and in bold. The translation stop and start codons are indicated in bold. Primers are indicated by arrows and by name, and the transcriptional start sites are indicated by “star burst” symbols above the nucleotide sequence. Intron/exon boundaries are indicated by vertical lines capped with a horizontal line (i.e., a “T” shaped symbol).

FIG. 2. T2 binding assay of XAGE-1 derived peptides. Three different peptides derived from XAGE-1 were examined for binding ability to HLA-A2 by using the T2 cell line (ATCC accession no. CRL-1992), which is a T-B lymphoblast fusion hybridoma cell line deficient in TAP1 and TAP2 gene expression. Cells were incubated with each peptide overnight before staining with an anti-HLA-A2 monoclonal antibody (BB7.2, from ATCC hybridoma no BB-82). FMP is a HLA-A2 binding peptide derived from influenza virus.

FIG. 3. CTLs induced with peptide of the invention can lyse human cancer cell expressing XAGE-1. Spleen cells derived from HLA-A2 transgenic mice immunized with a peptide of the invention (SEQ ID NO:6), as the effector cells, were stimulated in vitro for two weeks and used for a CTL assay. C1R.AAD cells pulsed or unpulsed with a peptide of SEQ ID NO:6 and a human osteosarcoma cell line expressing XAGE-1 (SB, described in Liu et al, Can Research 60:4752-4755 (2000)) were used as target cells. “E:T Ratio” stands for “Effector:Target ratio,” as indicated on the axis.

### DETAILED DESCRIPTION

### Introduction

A. Discovery that Xage-1 14 Peptide Induces CTL Response to Human XAGE-1-Expressing Cancers

The present invention relates to compositions and methods for enhancing the immune response of an individual to cells expressing an xage-1 protein, such as p9 or p16. peptide of fifty or fewer amino acids, the peptide comprising the sequence X₁X₂X₃PSAPSPX₄(SEQ ID NO:5), wherein: X₁ can be any amino acid; X₂ can be L, M, A, I, V, or T; X₃ can be a hydrophobic residue, methionine or alanine; and X₄ can be V, M, L, A, I, or T. In some embodiments, X₁ is tyrosine (SEQ ID NO:34), in some X₂ is leucine (SEQ ID NO:35) and in some X₃ is methionine (SEQ ID NO:36). In some embodiments, the peptide comprises an amino acid sequence selected from the group consisting of GVFPSAPSPV (SEQ ID NO:6), YVFPSAPSPV (SEQ ID NO:7), GLFPSAPSPV (SEQ ID NO:8), GVMPSAPSPV (SEQ ID NO:9), YLFPSAPSPV (SEQ ID NO:10), and GLMPSAPSPV (SEQ ID NO:11). In some embodiments, the peptide has an amino acid sequence selected from the group consisting of GVFPSAPSPV (SEQ ID NO:6), YVFPSAPSPV (SEQ ID NO:7), GLFPSAPSPV (SEQ ID NO:8), GVMPSAPSPV (SEQ ID NO:9), YLFPSAPSPV (SEQ ID NO:10), and GLMPSAPSPV (SEQ ID NO:11). In some embodiments, the T cells are isolated from a patient.

In another group of embodiments, the invention provides methods for stimulating or expanding T cells in vitro, comprising contacting said T cells with an antigen presenting cell pulsed with, transduced to express, or differentiated from a cell transduced with a nucleic acid encoding, an amino acid sequence of X₁X₂X₃PSAPSPX₄ (SEQ ID NO:5), wherein: X₁ can be any amino acid; X₂ can be L, M, A, I, V, or T; X₃ can be a hydrophobic residue, methionine or alanine; and X₄ can be V, M, L, A, I, or T. In some embodiments, X₁ is tyrosine (SEQ ID NO:34), in some X₂ is leucine (SEQ ID NO:35) and in some X₃ is methionine (SEQ ID NO:36). In some embodiments, the peptide comprises an amino acid sequence selected from the group consisting of GVFPSAPSPV (SEQ ID NO:6), YVFPSAPSPV (SEQ ID NO:7), GLFPSAPSPV (SEQ ID NO:8), GVMPSAPSPV (SEQ ID NO:9), YLFPSAPSPV (SEQ ID NO:10), and GLMPSAPSPV (SEQ ID NO:11). In some embodiments, the peptide has an amino acid sequence selected from the group consisting of GVFPSAPSPV (SEQ ID NO:6), YVFPSAPSPV (SEQ ID NO:7), GLFPSAPSPV (SEQ ID NO:8), GVMPSAPSPV (SEQ ID NO:9), YLFPSAPSPV (SEQ ID NO:10), and GLMPSAPSPV (SEQ ID NO:11).

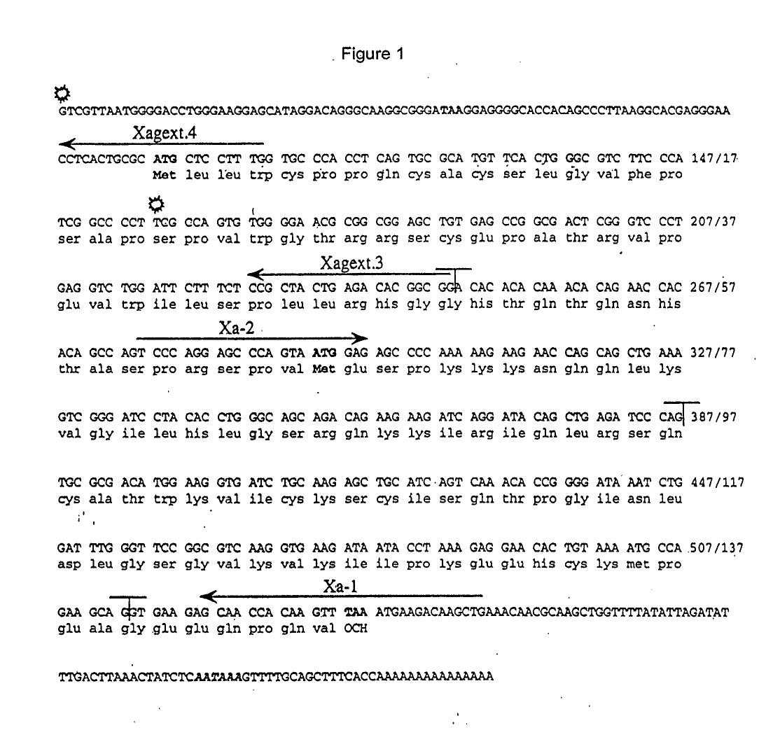

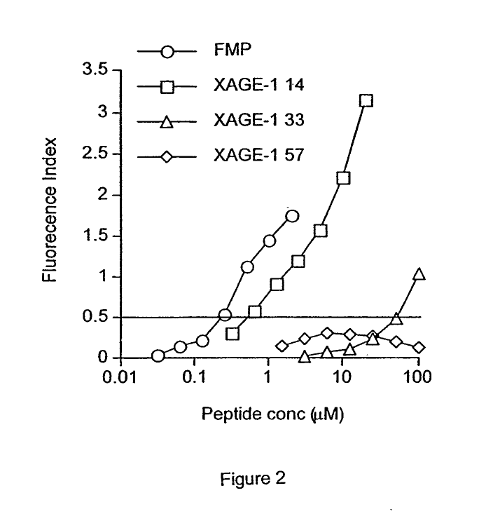

HLA-A2 is the most common human leukocyte antigen (“HLA”) Class 1 molecule in most of the world's population and is present in about 45% of the North American population. Surprisingly, it has now been discovered that, of the entire 16.3 kD protein encoded by RAGE-1, only a single, 10 amino acid sequence from the amino terminal end of the protein is efficient at binding HLA-A2. The peptide discovered to have this immunogenic activity is (in standard single letter code) GVFPSAPSPV (SEQ ID NO:6), which comprises residues 14-23 encoded by the first open reading frame of XAGE-1 (see FIG. 1). For convenience, the peptide has been designated as “xage-1 14.” As shown in FIG. 2, xage-1 14 showed surprisingly stronger binding to HLA-A2 compared to other candidate peptides.

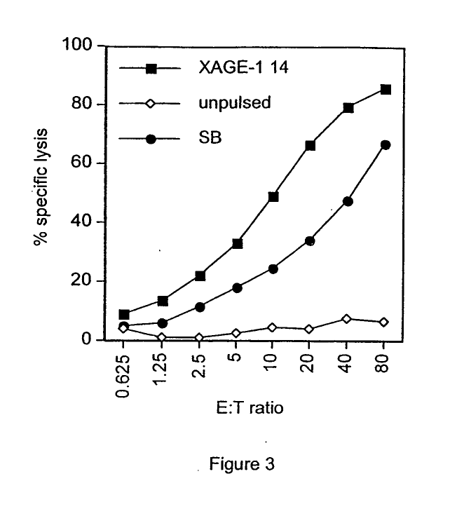

Even more importantly, the studies underlying the present invention demonstrate not only that xage-1 14 binds to HLA-A2, but also that animals immunized with xage-1 14 generate cytotoxic T lymphocytes (“CTLs”) which lyse human cancer cells expressing XAGE-1. CTLs from mice transgenic for human HLA-A2 and immunized with xage-1 14 were assayed for their ability to kill human cancer cells expressing XAGE-1. As shown in FIG. 3, the CTLs killed human RAGE-1-expressing cancer cells in a dose dependent manner. Moreover, these cell lysis assays demonstrate that proteins encoded by XAGE-1 are endogenously processed by XAGE-1 expressing cancer cells to present xage-1 14 on their surface in conjunction with HLA-A2.

Following the discovery of xage-1 14, a series of studies were undertaken. Surprisingly, these studies resulted in discovering variants of xage-1 14 which bound to HLA-A2 with affinities comparable to or greater than xage-1 14. For convenience, these peptides can be referred to by their respective substitutions compared to the sequence of xage-1 14 (SEQ ID NO:6, for example, the peptide designated as “1Y” below has a Tyrosine substituted at position 1 of SEQ ID NO:6, while “2L” has a Leucine at position 2), as set forth below:

| Name | Sequence | SEQ ID NO: |
|---|---|---|
| 1Y | YVFPSAPSPV | 7 |
| 2L | GLFPSAPSPV | 8 |
| 3M | GVMPSAPSPV | 9 |
| 1Y2L | YLFPSAPSPV | 10 |
| 2L3M | GLMPSAPSPV | 11 |

While all of these peptides are preferred, 3M xage-1 14 (SEQ ID NO:9) and 2L3M xage-1 14 (SEQ ID NO:11) have particularly high binding affinity to HLA-A2 and are more preferred, with 3M xage-1 14 (SEQ ID NO:9) being the most preferred.

While the studies underlying the present invention were conducted with human osteosarcoma cells as an exemplar XAGE-1-expressing cancer, it is expected that similar results will obtain for other XAGE-1 expressing cancers, such as XAGE-1-expressing prostate cancer, lung cancer, breast cancer, ovarian cancer, glioblastoma, pancreatic cancer, melanoma, and Ewing's sarcoma. The invention therefore provides important new in vitro and in vivo tools for inhibiting the growth of XAGE-1-expressing cancer cells.

### B. Peptides of the Invention

It is known in the art that peptides that bind to HLA-A2 typically are 9 to 10 amino acids in length. It is further known that, while the central residues of the peptides (residues 4-8, and residue 9 if the peptide is a 10-amino acid peptide) cannot be varied without some effect on binding or induction of CTL activity, some variations can be made with regard to residues 1-3 on the C-terminal end and with regard to residue 10 of the N-terminal end. The residues at positions 1, 2, 3 and at the last residue position (position 9 or 10, depending on the length of the peptide) are the ones that have been found to be permissive of certain types of variations. In general, position 1 can be any amino acid. Some literature indicates, however, that substituting tyrosine, Y, at position 1 results in a peptide with better binding to HLA-A2. Thus, Y is a preferred substitution at position 1 in peptides of the invention. Since G is the residue in this position in the native protein, G is also preferred at position 1.

It is also considered in the art that position 2 can be selected from the group consisting of L, M, A, I, V, and T, with L and M being preferred, and with L being particularly preferred. Since V is the residue in xage-1 14, it is also preferred. With respect to position 3, if position 3 is occupied by a hydrophobic residue (as in SEQ ID NO:6), if it is substituted, it is preferably substituted by another hydrophobic residue, such as W or Y. The present inventors, however, have also found that Alanine, A, and Methionine, M, can be advantageous at position 3, thus A and especially M are also preferred at this position. The last residue (X₄) is preferably aliphatic and may be V, M, L, A, I, and T, and is preferably V. Thus, the immunogenic peptides of this invention can be described by Formula I:

(SEQ ID NO: 5)
X₁X₂X₃PSAPSPX₄

wherein X₁ can be any amino acid and is preferably G or Y;
X₂ can be selected from the group consisting of L, M, A, I, V, and T, with L, V, and M being preferred;
X₃ can be a hydrophobic residue, A or M; and
X₄ may be V, M, L, A, I, or T, and is preferably V.

In addition to having a sequence as set forth in SEQ ID NO:5, the peptides of the invention bind to HLA-A2 and, when presented in conjunction with HLA-A2, induce cytotoxic T-cells to lyse cells expressing XAGE-1. Whether any particular peptide encompassed by SEQ ID NO:5 shares these characteristics can be readily determined by assays known in the art, such as those discussed herein. The peptides of SEQ ID NOs:6-11 are preferred, with the peptides of SEQ ID NOs:6, 9, and 11 being particularly preferred.

While peptides that bind to HLA-A2 are known to be 9 to 10 amino acids in length, persons of skill in the art will recognize that longer peptides can be processed by proteolytic cleavage within antigen presenting cells to create peptides of the correct length for HLA-A2 binding. Endogenous processing of this type, for example, results in presentation of xage-1 14 on cells of human XAGE-1-expressing cancers. While recombinant expression of peptides from XAGE-1 is possible, it is expected that the peptides of the invention will generally be made by synthetic techniques, which favor shorter peptides. Accordingly, it is anticipated that the peptides of the invention will typically be fifty or fewer amino acid residues in length and comprise a sequence of SEQ ID NO:5. Preferably, the peptides may be 40 or fewer residues, 30 or fewer residues, 20 or fewer residues, or 19, 18, 17, 16, 15, 14, 13, 12, or 11 residues, with each successively lower number of residues being more preferred. Even more preferred are 10-amino acid peptides of SEQ ID NO:6. Peptides of the sequence of SEQ ID NO:6, 9 and 11 are especially preferred. Peptides in which the residues designated as X₂ and X₄ in SEQ ID NO:5 are missing are generally disfavored, since they are considered anchor residues. A residue should be present in the position of X₁ since that provides the spatial relationship required for X₂.

As noted above, HLA-A2 can also bind to 9-amino acid peptides. As also noted, the core residues cannot be omitted without some effect on binding and CTL induction. Nonetheless, persons wishing to create a 9-mer from SEQ ID NO:5 can do so by omitting the proline at position 9 of SEQ ID NO:5, to create the sequence X₁X₂X₃PSAPSX₄(SEQ ID NO:38), or by omitting the serine at position 8 of SEQ ID NO:5 to create the sequence X₁X₂X₃PSAPPX₄ (SEQ ID NO:39). Less desirably, the proline at position 7 of SEQ ID NO:5 can be omitted, to create the sequence X₁X₂X₃PSASPX₄ (SEQ ID NO:40). These three sequences can be represented by the overall formula:

(SEQ ID NO: 41)
X₁X₂X₃PSA X₅ X₆ X₇X₄

wherein X₁₋₄ are as set forth above; X₅ is either proline or is absent; X₆ is either serine or is absent; and X₇ is either proline or is absent; provided that,
(i) when X₅ is absent, X₆ is serine and X₇ is proline;
(ii) when X₆ is absent, X₅ and X₇ are proline, and
(iii) when X₇ is absent, X₅ is proline and X₆ is serine.

It is anticipated that the peptides of SEQ ID NOS:38-40 will bind HLA-A2 with somewhat less affinity than will peptides of SEQ ID NO:5, and they are accordingly somewhat less preferred. As with the peptides of SEQ ID NO:5, to be considered as a peptide of the invention, the peptides of SEQ ID NOS:38-40 must bind HLA-A2 and, when used to immunize animals, the animals must generate cytotoxic T lymphocytes (“CTLs”) which lyse human cancer cells expressing XAGE-1. Assays for determining both of these things are known in the art. An exemplar assay by which it can be determined whether a given protein binds HLA-A2 is set forth in Example 1. An exemplar assay by which it can be determined whether a peptide used to immunize an animal causes generation of CTLs which lyse human cancer cells expressing XAGE-1 is set forth in Example 3.

C. Uses of the invention

The discovery of the peptides of the invention permits a number of in vitro and in vivo uses. For example, the peptides can be used ex vivo to stimulate cytotoxic T lymphocytes (CTLs) against cells expressing XAGE-1. The stimulated CTLs can then be used, for example, ex vivo to purge XAGE-1-expressing cancer cells from cell populations, such as bone marrow cells.

Alternatively, the stimulated CTLs can be infused into a patient (such as the patient from whom they were isolated) to augment the patient's immune response to an XAGE-1-expressing cancer. Isolation of CTLs from patients and the ex vivo expansion of CTL populations has been performed in the art for some time and has shown some success against various cancers. Based on the studies reported herein, it is expected that the use of the peptides of the invention will to stimulate CTL will result in a robust immune response that will inhibit the growth of cancer cells. In another alternative use, the peptides can be administered to persons with or at risk of a XAGE-1-expressing cancer to stimulate a CTL response against such cancers. In preferred uses, the peptides are administered to persons with a RAGE-1-expressing cancer. In preferred forms, the cancers are XAGE-1-expressing breast cancer, prostate cancer, lung cancer, ovarian cancer, glioblastoma, pancreatic cancer, melanoma, or Ewing's sarcoma, with breast cancer, prostate cancer, and Ewing's sarcoma particularly preferred. Uses of the peptides of the invention, of isolated nucleic acids encoding them, and of methods of making and administering them are described in more detail below.

### DEFINITIONS

Unless defined otherwise, all technical and scientific terms used herein have the meaning commonly understood by a person skilled in the art to which this invention belongs. The following references provide a general definition of many of the terms used in this invention: Singleton et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); THE GLOSSARY OF GENETICS, 5TH ED., R. Rieger et al. (eds.), Springer Verlag (1991); and Hale & Marham, THE HARPER COLLINS DICTIONARY OF BIOLOGY (1991). As used herein, the following terms have the meanings ascribed to them unless specified otherwise.

Reference to “XAGE-1” (that is, when printed in capital letters) refers to the RAGE-1 gene and “xage-1” (that is, when printed in lower case) refers to a protein encoded by the XAGE-1 gene.

“Xage-1 p9” and “p9” refer to a protein expressed from the XAGE-1 gene having a relative molecular weight of about 9 kD. The nucleic acid sequence (SEQ ID NO:1) encoding the xage-1 9 kD protein and the amino acid sequence (SEQ ID NO:2) of xage-1 p9, are set forth in FIG. 1. The nucleic acid sequence (SEQ ID NO:1) encoding the protein starts with nucleotide 281 of the nucleotide sequence shown in FIG. 1; the amino acid sequence (SEQ ID NO:2) starts at the methionine found at position 66 of the amino acid sequence shownin that Figure.

“Xage-1 p16” and “p16” refer to a protein expressed from the RAGE-1 gene having a calculated molecular weight of about 16.3 kD. The nucleic acid sequence encoding xage-1 p16 (SEQ ID NO:3) and the amino acid sequence of xage-1 p16 (SEQ ID NO:4), are set forth in FIG. 1. The nucleic acid sequence (SEQ ID NO:3) encoding the protein starts with nucleotide 1 of the nucleotide sequence shown in FIG. 1; the amino acid sequence (SEQ ID NO:4) starts at the methionine found at position 1 of the amino acid sequence in that Figure.

“Xage-1 14” refers to the amino acid sequence (SEQ ID NO:6) of residues 14-23 of the amino acid sequence (SEQ ID NO:4) of p16.

Use of the term “immunogenic” in connection with a composition, such as a peptide, indicates that the composition induces an immune response when administered to a mammal.

The expression that a cytotoxic T lymphocyte (CTL) is “specific for” a given peptide means that the CTL has a receptor that recognizes and binds a complex of the peptide and a major histocompatibility complex (MHC) molecule. With respect to HLA-A2, the MHC molecule is MHC class I.

The term “analog” includes any isolated polypeptide having an amino acid residue sequence substantially identical to the peptide sequence of SEQ ID NOs:5-11, in which one or more residues have been conservatively substituted with a functionally similar residue and which displays the functional aspects of the XAGE-1 peptides as described herein. Examples of conservative substitutions include the substitution of one non-polar (hydrophobic) residue such as isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid or another.

The phrase “conservative substitution” also includes the use of a chemically derivatized residue in place of a non-derivatized residue. “Chemical derivative” refers to a subject polypeptide having one or more residues chemically derivatized by reaction of a functional side group. Examples of such derivatized molecules include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzythistidine. Also included as chemical derivatives are those proteins or peptides which contain one or more naturally-occurring amino acid derivatives of the twenty standard amino acids. For examples: 4-hydroxyproline may be substituted for proline; 5-hydroxylysine may be substituted for lysine; 3-methylhistidine may be substituted for histidine; homoserine may be substituted for serine; and ornithine may be substituted for lysine.

In the context of comparing one polypeptide to another, “sequence identity is determined by comparing the sequence of xage-1, as the reference sequence, to a test sequence. Typically, the two sequences are aligned for maximal or optimal alignment.

A “ligand” is a compound that specifically binds to a target molecule.

A “receptor” is a compound that specifically binds to a ligand.

“Cytotoxic T lymphocytes” (“CTLs”) are important in the immune response to tumor cells. CTLs recognize peptide epitopes in the context of HLA class I molecules that are expressed on the surface of almost all nucleated cells.

Tumor-specific helper T lymphocytes (“HTLs”) are also known to be important for maintaining effective antitumor immunity. Their role in antitumor immunity has been demonstrated in animal models in which these cells not only serve to provide help for induction of CTL and antibody responses, but also provide effector functions, which are mediated by direct cell contact and also by secretion of lymphokines (e.g., IFNγ and TNF-α).

“Epitope” or “antigenic determinant” refers to a site on an antigen to which B and/or T cells respond. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in METHODS IN MOLECULAR BIOLOGY, Vol. 66, Glenn E. Morris, Ed (1996).

A ligand or a receptor “specifically binds to” a compound analyte when the ligand or receptor functions in a binding reaction which is determinative of the presence of the analyte in a sample of heterogeneous compounds. Thus, the ligand or receptor binds preferentially to a particular analyte and does not bind in a significant amount to other compounds present in the sample. For example, a polynucleotide specifically binds to an analyte polynucleotide comprising a complementary sequence and an antibody specifically binds under immunoassay conditions to an antigen analyte bearing an epitope against which the antibody was raised.

“Immunogenic peptide” refers to a peptide effective to induce an immune response in an organism. Immunogenic peptides can be used, for example, to raise an immune response against cells expressing a protein in which the immunogenic peptide is present.

An “immunogenic amount” is an amount effective to elicit an immune response in a subject.

The term “contacting” includes reference to placement in direct physical association.

An “expression plasmid” comprises a nucleotide sequence encoding a molecule or interest, which is operably linked to a promoter.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer. The term “protein” typically refers to large polypeptides. The term “peptide” typically refers to short polypeptides. For purposes of this invention, peptides are those molecules comprising up to 50 amino acid residues, and proteins comprise 50 or more amino acid residues. Methods of synthesis and/or delivery of peptides and proteins of the invention are, however, similar, if not identical, as will be appreciated by one of skill in the art. Therefore, where appropriate, these terms are synonymous when discussing methods of synthesis, modification or use as therapeutic or diagnostic reagents.

“Amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and o-phosphoserine. “Amino acid analog” refers to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Amino acid sequence” refers to the positional relationship of amino acid residues as they exist in a given polypeptide or protein. Conventional notation is used herein to describe amino acid sequences: the left-hand end of a peptide or protein is the amino end or terminus and the right-hand end is the carboxyl end or terminus.

“Fusion protein” refers to a polypeptide formed by the joining of two or more polypeptides through a peptide bond formed by the amino terminus of one polypeptide and the carboxyl terminus of the other polypeptide. A fusion protein may is typically expressed as a single polypeptide from a nucleic acid sequence encoding the single contiguous fusion protein. However, a fusion protein can also be formed by the chemical coupling of the constituent polypeptides.

“Fusion molecules” refers to any molecule formed through the structural linkage of a peptide of the present invention to one or more molecules, particularly macromolecules. In the context of the present invention, other molecules that can be joined to peptides of the invention to form fusion molecules include sugars and polysaccharides, other peptides and proteins, lipids, and nucleotides and nucleic acids.

“Antibody” refers to a polypeptide ligand comprising at least a light chain or heavy chain immunoglobulin variable region which specifically recognizes and binds an epitope (e.g., an antigen). This includes intact immunoglobulins and the variants and portions of them well known in the art such as, Fab′ fragments, F(ab)′₂ fragments, single chain Fv proteins (“scFv”), and disulfide stabilized Fv proteins (“dsFv”). An scFv protein is a fusion protein in which a light chain variable region of an immunoglobulin and a heavy chain variable region of an immunoglobulin are bound by a linker. The term also includes genetically engineered forms such as chimeric antibodies (e.g., humanized murine antibodies), heteroconjugate antibodies (e.g., bispecific antibodies). See also, Pierce Catalog and Handbook, 1994-1995 (Pierce Chemical Co., Rockford, Ill.); Kuby, J., Immunology, 3^{rd} Ed., W.H. Freeman & Co., New York (1997).

An antibody immunologically reactive with a particular antigen can be generated by recombinant methods such as selection of libraries of recombinant antibodies in phage or similar vectors, see, e.g., Huse, et al., Science 246:1275-1281 (1989); Ward, et al., Nature 341:544-546 (1989); and Vaughan, et al., Nature Biotech. 14:309-314 (1996), or by immunizing an animal with the antigen or with DNA encoding the antigen.

As used herein, the term “anti-xage-1” in reference to an antibody, includes reference to an antibody which is generated against xage-1 p9 or xage-1 p16. The antibody can be generated against human xage-1 p9 or p16 synthesized by a mammal after introduction into the animal of cDNA which encodes a human xage-1 protein.

“Immunoassay” refers to a method of detecting an analyte in a sample in which specificity for the analyte is conferred by the specific binding between an antibody and a ligand. This includes detecting an antibody analyte through specific binding between the antibody and a ligand. See Harlow and Lane (1988) ANTIBODIES, A LABORATORY MANUAL, Cold Spring Harbor Publications, New York, for a description of immunoassay formats and conditions that can be used to determine specific immunoreactivity.

“Conservative substitution” refers to the substitution in a polypeptide of an amino acid with a functionally similar amino acid. The following six groups each contain amino acids that are conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).
   See also, Creighton, PROTEINS, W.H. Freeman and Company, New York (1984).

Two proteins are “homologs” of each other if they exist in different species, are derived from a common genetic ancestor and share at least 70% amino acid sequence identity.

“Substantially pure” or “isolated” means an object species is the predominant species present (i.e., on a molar basis, more abundant than any other individual macromolecular species in the composition), and a substantially purified fraction is a composition wherein the object species comprises at least about 50% (on a molar basis) of all macromolecular species present. Generally, a substantially pure composition means that about 80% to 90% or more of the macromolecular species present in the composition is the purified species of interest. The object species is purified to essential homogeneity (contaminant species cannot be detected in the composition by conventional detection methods) if the composition consists essentially of a single macromolecular species. Solvent species, small molecules (<500 Daltons), stabilizers (e.g., BSA), and elemental ion species are not considered macromolecular species for purposes of this definition.

“Nucleic acid” refers to a polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Thus, the term includes nucleotide polymers in which the nucleotides and the linkages between them include non-naturally occurring synthetic analogs, such as, for example and without limitation, phosphorothioates, phosphoramidates, methyl phosphonates, chiral-methyl phosphonates, 2-O-methyl ribonucleotides, peptide-nucleic acids (PNAs), and the like. Such polynucleotides can be synthesized, for example, using an automated DNA synthesizer. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe nucleotide sequences: the left-hand end of a single-stranded nucleotide sequence is the 5′-end; the left-hand direction of a double-stranded nucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand having the same sequence as an mRNA transcribed from that DNA and which are located 5′ to the 5′-end of the RNA transcript are referred to as “upstream sequences”; sequences on the DNA strand having the same sequence as the RNA and which are 3′ to the 3′ end of the coding RNA transcript are referred to as “downstream sequences.”

“cDNA” refers to a DNA that is complementary or identical to an mRNA, in either single stranded or double stranded form.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Recombinant nucleic acid” refers to a nucleic acid having nucleotide sequences that are not naturally joined together. This includes nucleic acid vectors comprising an amplified or assembled nucleic acid which can be used to transform a suitable host cell. A host cell that comprises the recombinant nucleic acid is referred to as a “recombinant host cell.” The gene is then expressed in the recombinant host cell to produce, e.g., a “recombinant polypeptide.” A recombinant nucleic acid may serve a non-coding function (e.g., promoter, origin of replication, ribosome-binding site, etc.) as well.

“Expression control sequence” refers to a nucleotide sequence in a polynucleotide that regulates the expression (transcription and/or translation) of a nucleotide sequence operatively linked thereto. “Operatively linked” refers to a functional relationship between two parts in which the activity of one part (e.g., the ability to regulate transcription) results in an action on the other part (e.g., transcription of the sequence). Expression control sequences can include, for example and without limitation, sequences of promoters (e.g., inducible or constitutive), enhancers, transcription terminators, a start codon (i.e., ATG), splicing signals for introns, and stop codons.

“Expression cassette” refers to a recombinant nucleic acid construct comprising an expression control sequence operatively linked to an expressible nucleotide sequence. An expression cassette generally comprises sufficient cis-acting elements for expression; other elements for expression can be supplied by the host cell or in vitro expression system.

“Expression vector” refers to a vector comprising an expression cassette. Expression vectors include all those known in the art, such as cosmids, plasmids (e.g., naked or contained in liposomes) and viruses that incorporate the expression cassette.

A first sequence is an “antisense sequence” with respect to a second sequence if a polynucleotide whose sequence is the first sequence specifically hybridizes with a polynucleotide whose sequence is the second sequence.

Terms used to describe sequence relationships between two or more nucleotide sequences or amino acid sequences include “reference sequence,” “selected from,” “comparison window,” “identical,” “percentage of sequence identity,” “substantially identical,” “complementary,” and “substantially complementary.”

For sequence comparison of nucleic acid sequences, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters are used. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds 1995 supplement)).

One example of a useful algorithm is PILEUP. PILEUP uses a simplification of the progressive alignment method of Feng & Doolittle, J. Mol. Evol. 35:351-360 (1987). The method used is similar to the method described by Higgins & Sharp, CABIOS 5:151-153 (1989). Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., Nuc. Acids Res. 12:387-395 (1984).

Another example of algorithms that are suitable for determining percent sequence identity and sequence similarity are the BLAST and the BLAST 2.0 algorithm, which are described in Altschul et al., J. Mol. Biol. 215:403-410 (1990) and Altschul et al., Nucleic Acids Res. 25:3389-3402 (1977)). Software for performing BLAST analyses is publicly available through the National Center for Biotechnology Information (http://www.ncbi.nlm.nih.gov/). The BLASTN program (for nucleotide sequences) uses as defaults a word length (W) of 11, alignments (B) of 50, expectation (E) of 10, and a comparison of both strands. The BLASTP program (for amino acid sequences) uses as defaults a word length (W) of 3, and expectation (E) of 10, and the BLOSUM62 scoring matrix (see Henikoff & Henikoff, Proc. Natl. Acad. Sci. USA 89:10915 (1989)).

“Stringent hybridization conditions” refers to 50% formamide, 5×SSC and 1% SDS incubated at 42° C. or 5×SSC and 1% SDS incubated at 65° C., with a wash in 0.2×SSC and 0.1% SDS at 65° C.

“Naturally-occurring” as applied to an object refers to the fact that the object can be found in nature. For example, an amino acid or nucleotide sequence that is present in an organism (including viruses) that can be isolated from a source in nature and which has not been intentionally modified by man in the laboratory is naturally-occurring.

“Linker” refers to a molecule that joins two other molecules, either covalently, or through ionic, van der Waals or hydrogen bonds, e.g., a nucleic acid molecule that hybridizes to one complementary sequence at the 5′ end and to another complementary sequence at the 3′ end, thus joining two non-complementary sequences.

“Pharmaceutical composition” refers to a composition suitablefor pharmaceutical use in a mammal. A pharmaceutical composition comprises a pharmacologically effective amount of an active agent and a pharmaceutically acceptable carrier.

“Pharmacologically effective amount” refers to an amount of an agent effective to produce the intended pharmacological result.

“Pharmaceutically acceptable carrier” refers to any of the standard pharmaceutical carriers, buffers, and excipients, such as a phosphate buffered saline solution, 5% aqueous solution of dextrose, and emulsions, such as an oil/water or water/oil emulsion, and various types of wetting agents and/or adjuvants. Suitable pharmaceutical carriers and formulations are described in REMINGTON's PHARMACEUTICAL SCIENCES, 19th Ed. (Mack Publishing Co., Easton, 1995). Preferred pharmaceutical carriers depend upon the intended mode of administration of the active agent. Typical modes of administration include enteral (e.g., oral) or parenteral (e.g., subcutaneous, intramuscular, intravenous or intraperitoneal injection; or topical, transdermal, or transmucosal administration). A “pharmaceutically acceptable salt” is a salt that can be formulated into a compound for pharmaceutical use including, e.g., metal salts (sodium, potassium, magnesium, calcium, etc.) and salts of ammonia or organic amines.

A “subject” of diagnosis or treatment can be a human or a non-human mammal.

“Administration” of a composition refers to introducing the composition into the subject by a chosen route of administration. For example, if the chosen route is intravenous, the composition is administered by introducing the composition into a vein of the subject.

“Treatment” refers to prophylactic treatment or therapeutic treatment.

A “prophylactic” treatment is a treatment administered to a subject who does not exhibit signs of a disease or exhibits only early signs for the purpose of decreasing the risk of developing pathology.

A “therapeutic” treatment is a treatment administered to a subject who exhibits signs of pathology for the purpose of diminishing or eliminating those signs.

“Diagnostic” means identifying the presence or nature of a pathologic condition. Diagnostic methods differ in their sensitivity and specificity. The “sensitivity” of a diagnostic assay is the percentage of diseased individuals who test positive (percent of true positives). The “specificity” of a diagnostic assay is 1 minus the false positive rate, where the false positive rate is defined as the proportion of those without the disease who test positive. While a particular diagnostic method may not provide a definitive diagnosis of a condition, it suffices if the method provides a positive indication that aids in diagnosis.

“Prognostic” means predicting the probable development (e.g., severity) of a pathologic condition.

### Peptides of the Invention

T-lymphocytes recognize antigen in association with Class I or Class II MHC molecules in the form of a peptide fragment bound to an MHC molecule. The degree of peptide binding to a given MHC allele is based on amino acids at particular positions within the peptide (Parker et al. (1992) Journal of Immunology 149:3580; Kubo, et al. (1994) Journal of Immunology 52:3913-3924; Ruppert J. et al. (1993) Cell 74:929-937; Falk et al. (1991) Nature 351:290-296). Therefore, another embodiment of this invention relates to peptides derived from SEQ ID NOS:5 or 6 which have been modified to increase immunogenicity by enhancing binding of the peptide to the MHC molecule with which the peptide is associated. By way of example, modification may include substitution, deletion or addition of an amino acid in the given immunogenic peptide sequence or mutation of existing amino acids within the given immunogenic peptide sequence, or derivatization of existing amino acids within the given immunogenic peptide sequence. Any amino acid in the immunogenic peptide sequence may be modified in accordance with this invention. In a preferred embodiment at least one amino acid is substituted or replaced within the given immunogenic peptide sequence. Any amino acid may be used to substitute or replace a given amino acid within the immunogenic peptide sequence, however, certain types of residues are preferred at certain positions, as indicated in the formula set forth in the Introduction. Modified peptides are intended to include any immunogenic XAGE-1 peptide which has been modified and exhibits enhanced binding to the MHC molecule with which it associates when presented to the T-cell.

By way of example, the HLA-A2 allele binds peptides of nine or ten amino acids. Examples of positions within the peptide that may be altered to enhance binding include, but are not limited to, the first position, the second position, the third position and the last position of the peptide. Any amino acid may be used to substitute or replace these positions within the immunogenic peptide sequence. For enhanced binding to HLA-A2 the amino acid at the second position of the peptide is preferably a hydrophobic aliphatic amino acid. Examples of amino acids that may be used at the second position include, but are not limited to, leucine, methionine, alanine, isoleucine, valine, and threonine. More preferred peptides have a leucine, valine, or methionine at position 2.

The last amino acid of the peptide (either the 9th or 10th amino acid depending on the length of the peptide) is preferably a hydrophobic aliphatic amino acid. Examples of amino acids that may be used in the last position of the peptide include, but are not limited to, valine, methionine, leucine, alanine, isoleucine, or threonine. Preferably, valine is at the last position in the peptide. The amino acids at the first and third positions in the peptide may also be modified to enhance binding of the peptide to the MHC Class I molecule. The amino acids at the first and third positions in the peptide may be any amino acid, but are preferably hydrophobic aliphatic amino acids or aromatic amino acids. Examples of amino acids that may be used at these positions include, but are not limited to, leucine, methionine, valine, alanine, isoleucine, threonine, tryptophan, phenylalanine, tyrosine, serine, aspartic acid, or lysine. Tyrosine (Y) is preferred at position 1 to increase binding. In some embodiments, methionine or alanine is preferred at position 3.

Examples of modified xage-1 14 peptides include, but are not limited to, GVWPSAPSPV (SEQ ID NO: 12), GVYPSAPSPV (SEQ ID NO:13) and TVWPSAPSPM (SEQ ID NO: 14), SMYPSAPSPI (SEQ ID NO: 15); SVFPSAPSPT (SEQ ID NO:16); GVWPSAPSPM (SEQ ID NO:17); SVWPSAPSPV (SEQ ID NO:18); GLWPSAPSPV (SEQ ID NO:19); IVWPSAPSPV (SEQ ID NO:20); GLAPSAPSPV (SEQ ID NO:21); GVAPSAPSPV (SEQ ID NO:22); YLFPSAPSPM (SEQ ID NO:23); YLAPSAPSPI (SEQ ID NO:24); and YLAPSAPSPV (SEQ ID NO:25).

The 9-mer peptides of SEQ ID NOS:38-40 can be varied in the same ways described for the peptides of SEQ ID NO:5.

This invention further includes analogs of immunogenic modified peptides derived from the xage-1 14 (e.g., SEQ ID NOS:5-25) which have been modified. The term analog is intended to encompass peptides which display the functional aspects of these modified peptides. The term analog also includes conservative substitutions or chemical derivatives of these modified peptides as described above. These modified peptides may be synthetically or recombinantly produced by conventional methods. To be considered a peptide of the invention, the peptide, whether modified or not, must bind HLA-A2 and elicit a CTL response. Determining whether any particular peptide elicits a CTL response can be performed by assays, as known in the art and as described below.

### Nucleic Acids Encoding Peptides of the Invention

In some embodiments, isolated nucleic acids encoding the peptides of the invention are used with or instead of the peptides of the invention. For example, DNA can be injected into the skin of a subject, where it is taken up and expressed by antigen presenting cells present in the skin. Conveniently, the nucleic acid is operably linked to a promoter to facilitate expression. The nucleic acids can also be used in expression cassettes to facilitate recombinant expression of the peptides in appropriate expression systems.

In a preferred embodiment, the nucleic acid sequence encoding SEQ ID NO:6 is the native sequence, which is set forth in FIG. 1:

(SEQ ID NO: 26)
GGCGTCTTCCCATCGGCCCCTTCGCCAGTG

Persons of skill will appreciate, however, that, due to the degeneracy of the genetic code, any particular peptide of the invention can be encoded by a multitude of nucleic acid sequences. Thus, for example, the glycine in position 1 of SEQ ID NO:6 can be encoded by GGC, as set forth above, or by any of three other codons: GGT, GGA, or GGG, any of which substituted into the sequence of SEQ ID NO:26 will result in the expression of the same protein. Each of these variations, and the similar substitutions that can be made in the codons for the other amino acid residues of SEQ ID NO:6, are contemplated and are included in the scope of the invention.

As noted in the Introduction to the Detailed Description, several peptides, SEQ ID NOS:7-11, were derived from SEQ ID NO:6 and found to have high binding for HLA-A2. For these peptides, it is desirable if they have the sequence of SEQ ID NO:26, except for the codon encoding the residue or residues of the sequence by which that sequence differs from SEQ ID NO:6. For example, in SEQ ID NO:9, the phenylalanine at the third position of SEQ ID NO:6 is replaced by a methionine. Therefore, in a preferred embodiment, the nucleic acid sequence encoding the “3M xage-1 14” peptide of SEQ ID NO:9 is identical to that of SEQ ID NO:26, except for the substitution of the codon that encodes methionine (methionine is encoded by only one codon) for the codon encoding phenylalanine in SEQ ID NO:26. Thus, in a preferred form, the nucleic acid sequence encoding SEQ ID NO:9 is:

(SEQ ID NO: 27)
GGCGTCATGCCATCGGCCCCTTCGCCAGTG.

Similarly, in a preferred embodiment, the nucleic acid sequence encoding the “2L3M xage-1 14” peptide of SEQ ID NO:11 is identical to that of SEQ ID NO:26, except for the substitution of a codon encoding leucine for that encoding valine at position 2 of SEQ ID NO:6 and of the codon encoding methionine for the codon encoding phenylalanine at position 3 of SEQ ID NO:6. Thus, in a preferred form, the nucleic acid sequence encoding SEQ ID NO:11 is

(SEQ ID NO: 28)
GGCCTTATGCCATCGGCCCCTTCGCCAGTG

Because leucine is also encoded by three other codons, however, SEQ ID NO:11 could just as easily be encoded by any the following sequences, each of which is also preferred:

(SEQ ID NO: 29)
GGCCTCATGCCATCGGCCCCTTCGCCAGTG;
(SEQ ID NO: 30)
GGCCTAATGCCATCGGCCCCTTCGCCAGTG
(SEQ ID NO: 31)
GGCCTGATGCCATCGGCCCCTTCGCCAGTG

Once again, because of the degeneracy of the genetic code, a multitude of other nucleic acid sequences could be used to encode the peptides of the invention. For example, valine is encoded by four separate codons: GTT, GTC, GTA, and GTG. Thus, the valine encoded by “GTG” at the end of each of the sequences set forth above could be encoded instead by GTT, GTC, or GTA. Similarly, proline is encoded by four different codons. Thus, each of the prolines encoded by the codon “CCA” in the sequences above could independently be encoded instead by any of the other three codons encoding proline. Thus, persons of skill will appreciated that the nucleic acid sequences set forth above are illustrative of sequences that can encode the peptides of the invention, rather then limiting.

Similarly, the 9-mer peptides described by SEQ ID NO:41 can be encoded by a multitude of nucleic acids. In some embodiments, the nucleic acids have the native sequence of xage-1 14, except of course for the omission of the codon which would otherwise encode the omitted residue of position 7, 8, or 9. For example, an exemplar nucleic acid encoding a peptide of SEQ ID NO:39 is GGCGTCTTCCCATCGGCCCCTTCGGTG (SEQ ID NO:42), while an exemplar nucleic acid encoding a peptide of SEQ ID NO:38 is GGCGTCTTCCCATCGGCCCCTCCAGTG (SEQ ID NO:43) and an exemplar nucleic acid encoding a peptide of SEQ ID NO:40 is GGCGTCTTCCCATCGGCCTCGCCAGTG (SEQ ID NO:44). As already noted, because of the degeneracy of the genetic code, a multitude of other nucleic acid sequences could be used to encode the 9-mer peptides of the invention. Thus, persons of skill will appreciated that the nucleic acid sequences set forth above are merely illustrative of sequences that can encode the 9-mer peptides of the invention.

### Methods of Testing for Immunogenic Capability of a Peptide

To ensure that a peptide of the invention elicits a CTL response to XAGE-1-expressing cancer cells in vivo, the peptide may be used to immunize T cells in vitro from individuals of the appropriate HLA allele. Thereafter, the immunized cells' capacity to induce lysis of XAGE-1-expressing target cells is evaluated.

Conventional assays utilized to detect T cell responses include proliferation assays, lymphokine secretion assays, direct cytotoxicity assays, and limiting dilution assays. For example, antigen-presenting cells that have been incubated with a peptide can be assayed for the ability to induce CTL responses in responder cell populations.

PBMCs may be used as the responder cell source of CTL precursors. The appropriate antigen-presenting cells are incubated with peptide, after which the peptide-loaded antigen-presenting cells are then incubated with the responder cell population under optimized culture conditions. Positive CTL activation can be determined by assaying the culture for the presence of CTLs that kill radio-labeled target cells, both specific peptide-pulsed targets as well as target cells expressing endogenously processed forms of the antigen from which the peptide sequence was derived.

Alternatively, mutant mammalian cell lines that are deficient in their ability to load class I molecules with internally processed peptides, such as the mouse cell lines RMA-S (Karre, et al. Nature, 319:675 (1986); Ljunggren, et al., Eur. J. Immunol. 21:2963-2970 (1991)), and the human somatic T cell hybridoma, T-2 (Cerundolo, et al., Nature 345:449-452 (1990)) and that have been transfected with the appropriate human class I genes may be conveniently used. To test for the capacity of an immunogenic peptide of the invention to induce in vitro primary CTL response, the peptide is added to the cells. Other eukaryotic cell lines which could be used include various insect cell lines such as mosquito larvae (ATCC cell lines CCL 125, 126, 1660, 1591, 6585, 6586), silkworm (ATTC CRL 8851), armyworm (ATCC CRL 1711), moth (ATCC CCL 80) and Drosophila cell lines such as a Schneider cell line (see Schneider J. Embryol. Exp. Morphol. 27:353-365 (1927)) that have been transfected with the appropriate human class I MHC allele encoding genes and the human B₂ microglobulin genes.

One preferred type of mutant cell line for determining ability of peptides to bind to MHC I molecules are cells with impaired transporter associated with antigen processing (TAP) function. Impaired TAP function results in expression of low levels of cell surface major histocompatibility complex (MHC) class I molecules. Cells with impaired TAP function are generally resistant to lysis by MHC class I restricted cytotoxic T lymphocytes (CTLs). E.g., Wolpert et al., PNAS 94(21):11496-11501 (1997). T2 cells are a human, TAP-deficient B lymphoblastic cell line available from the ATCC under accession number CRL-1992. According to the ATCC, “[t]he cells do not express HLA DR and are Class II major histocompatibility (MHC) antigen negative. The cells synthesize, but do not express, HLA B5.” The cell line is useful for studying T cell recognition of class I major histocompatibility antigens because it is deficient in the transport and presentation of endogenous peptides. Candidate peptides can be incubated with T2 cells to determine if they bind to MHC I molecules, such as HLA-A2. T2 cells to which the peptides have not bound show limited presence of HLA-A2 when contacted with labeled anti-human HLA-A2 antibodies. Cells to which the peptides have bound to HLA-A2 show increased amounts of label compared to cells not contacted with the peptide.

A method which allows direct quantification of antigen-specific T cells is staining with Fluorescein-labeled HLA tetrameric complexes (Altman et al., Proc. Natl. Acad. Sci. USA 90:10330 (1993); Altman et al., Science 274:94 (1996)). Alternatively, staining for intracellular lymphokines, interferon-γ release assays or ELISPOT assays, can be used to evaluate T-cell responses.

CTL activation may be assessed using such techniques known to those in the art such as T cell proliferation and secretion of lymphokines, e.g. IL-2 (see, e.g. Alexander et al., Immunity 1:751-761 (1994)).

In another method, IFN-γ and/or RANTES production by stimulated T cells can be measured in the T cell culture supernatant. Methods fo'r measuring CTL response, RANTES and IFN-γ production of stimulated T cells are well known in the art

### Methods of Eliciting a Cell-Mediated Immune Response Against Cells Expressing XAGE-1

XAGE-1 is expressed by cells of a number of cancers, including cancers of the prostate, breast, ovaries, lung and pancreas, in addition to some muscle and bone cancers. Therefore, XAGE-1 can be used as a target of intervention in inhibiting the growth of cells of these cancers which express XAGE-1, as well as a marker for cancer cells that have metastasized from these cancers. This invention provides compositions and methods of inhibiting the growth of these cancers. When used prophylatically, the compositions and methods can prevent or slow the development of XAGE-1-expressing cancers by enhancing the subject's own immune response to such cancers. When used therapeutically, the compositions and methods can be used to inhibit the growth of a diagnosed XAGE-1-expressing cancer. The methods involve immunizing a subject with one or more peptides of SEQ ID NOS:5-11 or SEQ ID NOS:38-40, or with a nucleic acid encoding one or more peptides of SEQ ID NOS:5-11 or SEQ ID NOS:38-40, thereby eliciting a cell-mediated immune response against cells expressing XAGE-1.

In another set of methods of the invention, hematopoietic stem cells from a patient with an XAGE-1 expressing cancer, or considered at risk for developing such a cancer (such as a person who has previously had breast cancer or who has a genetic high risk profile) are transduced ex vivo with a vector encoding a peptide of SEQ ID NOS:5-11 or of SEQ ID NOS:38-40 and differentiated into dendritic cells expressing a peptide of the invention, as taught in, e.g., Hwu et al., International Publication WO 97/20263. T cells from the patient are then contacted with the differentiated dendritic cells, expanded in vitro, and then infused into the patient to increase the patient's immune response. Dendritic cells are recognized as being among the most effective antigen presenting cells (APCs).

Similarly, T cells from the patient can be contacted with the peptides of the invention, expanded ex vivo, and then infused into the patient to augment the patient's response to an XAGE-1-expressing cancer. For example, T cells may be isolated from bone marrow, peripheral blood, or a fraction of bone marrow or peripheral blood of a patient, using a commercially available cell separation system, such as the Isolex™ System, available from Nexell Therapeutics, Inc. (Irvine, Calif.). Cell separators and procedures for separation are also taught, for example, in U.S. Pat. Nos. 5,240,856; 5,215,926; and in WO 89/06280; WO 91/16116 and WO 92/07243). Alternatively, T cells may be derived from related or unrelated humans, non-human mammals, cell lines or cultures.

The T cells may be stimulated with a polypeptide of the invention, or an APC that expresses such a polypeptide. Such stimulation is performed under conditions and for a time sufficient to permit the generation of T cells that are specific for the polypeptide. T cells are considered to be specific for a peptide of the invention if the T cells specifically proliferate, secrete cytokines or kill target cells coated with the polypeptide or expressing a gene encoding the polypeptide. T cell specificity may be evaluated using any of a variety of standard techniques. For example, within a chromium release assay or proliferation assay, a stimulation index of more than two fold increase in lysis and/or proliferation, compared to negative controls, indicates T cell specificity. Such assays may be performed, for example, as described in Chen et al., Cancer Res. 54:1065-1070, 1994. Alternatively, detection of the proliferation of T cells may be accomplished by a variety of known techniques, as discussed elsewhere herein.

For therapeutic purposes, CD4⁺ or CD8⁺ T cells that proliferate in response to a peptide of the invention, polynucleotide encoding a peptide of the invention or APC presenting a peptide of the invention in conjunction with an MHC Class I molecule can be expanded in number either in vitro or in vivo. Proliferation of such T cells in vitro may be accomplished in a variety of ways. For example, the T cells can be re-exposed to a peptide of the invention, with or without the addition of T cell growth factors, such as interleukin-2. Alternatively, one or more T cells that proliferate in the presence of a peptide of the invention can be expanded in number by cloning. Methods for cloning cells are well known in the art, and include limiting dilution.

Immunization with the compositions of the invention can be active or passive. In active immunization, the immune response is elicited in the subject in vivo. In passive immunization, T cells activated against the polypeptide are cultured in vitro and administered to the subject.

In male subjects, such methods may be expected to result in the destruction of healthy testis tissue that expresses RAGE-1. The testes are not, however, an essential organ. Loss of the testes must be balanced against the chance for loss of the subject's life from the cancer, and the testes may, indeed, be surgically removed prior to institution of immunotherapy. Such orchiectomy is indeed one treatment for prostate cancer, one of the targets of immunotherapy of this invention.

Molecules with high levels of sequence identity to SEQ ID NOS:6-11 or SEQ ID NOS:38-40 are also useful to elicit an immune response. Such molecules can be recognized as “foreign” to the immune system, yet generate antibodies or CTLs that cross react with XAGE-1 proteins. Analogs of SEQ ID NO:6-11 or SEQ ID NOS:38-40 whose amino acid sequences are at least 90% identical and which activate T-lymphocytes to cells which express RAGE-1 are also useful.

The application of these molecules is now described. These methods are also described in Rosenberg et al., Immunol. Today 1997 18:175 (1997) and Restifo et al., Oncology 11:50 (1999).

One method of invoking an immune response involves immunizing the subject with a polypeptide of SEQ ID NOS:5-11 or SEQ ID NOS:38-40, either alone or, preferably, combined with an immunogenic molecule or composition, such as an adjuvant. Adjuvants such as Freund's incomplete adjuvant, lipids and liposomes, gp96, Hsp70 and Hsp90, are known in the art. In some embodiments, the adjuvant preferentially stimulates a cell mediated immune response. This is often described as a TH1 response, as opposed to a TH2 response, which describes humoral, antibody based responses. TH1 responses are associated with the production of interferon-γ and IL-2, while TH2 responses are associated with the production of IL-4, IL-5, IL-6 and IL-10.

Adjuvants that preferentially induce TH1 responses are known in the art. A number of such adjuvants are described, for example, in U.S. Pat. No. 6,451,320. The '320 patent, for examples, names as such an adjuvant 3 De-O-acylated monophosphoryl lipid A (3D-MPL), which it notes is set forth in GB 2220211. Chemically, it is a mixture of 3 De-O-acylated monophosphoryl lipid A with 4, 5 or 6 acylated chains and is manufactured by Ribi Immunochem, Montana. The patent states that a preferred form of 3 De-O-acylated monophosphoryl lipid A is disclosed in European Patent 0 689 454 B1 (SmithKline Beecham Biologicals SA). The patent further names as such an adjuvant QS21, an HPLC-purified non-toxic fraction derived from the bark of Quillaja saponaria Molina, described in U.S. Pat. No. 5,057,540, and immunomodulatory oligonucleotides, for example unmethylated CpG sequences as disclosed in WO 96/02555. Other adjuvants are described in International Publication Nos. WO 94/00153 and WO 95/17209.

Combinations of different TH1 stimulating adjuvants, such as those mentioned above, are also contemplated as providing an adjuvant which is a preferential stimulator of TH1 cell response. For example, QS21 can be formulated together with 3D-MPL. The '320 patent indicates that the ratio of QS21:3 D-MPL will typically be in the order of 1:10 to 10:1; preferably 1:5 to 5:1 and often substantially 1:1. Preferred ranges are 2.5:1 to 1:1 3D-MPL: QS21. Preferably a carrier is also present in the composition. The carrier may be an oil in water emulsion, or an aluminum salt, such as aluminum phosphate or aluminum hydroxide. A preferred oil-in-water emulsion comprises a metabolisible oil, such as squalene, alpha tocopherol and Tween® 80. Additionally the oil in water emulsion may contain lecithin or tricaprylin.

Typically for human administration QS21 and 3D-MPL are present in the range of 1 μg-200 μg, such as 10-100 μg, preferably 10 μg-50 μg per dose. Typically the oil in water will comprise from 2 to 10% squalene, from 2 to 10% alpha tocopherol and from 0.3 to 3% Tween® 80. The ratio of squalene:alpha tocopherol may be equal to or less than 1 as this provides a more stable emulsion. Surfactants such as sorbitan trioleate may also be present at a level of 1%. In some cases it may be advantageous that the compositions further contain a stabiliser. Non-toxic oil in water emulsions may contain a non-toxic oil, e.g. squalane or squalene, and an emulsifier, such as Tween® 80, in an aqueous carrier. The aqueous carrier may be, for example, phosphate buffered saline.

In a preferred embodiment, the adjuvant is soluble recombinant human GM-CSF, which is typically administered in a dose of 100 μg.

Peptides of the invention may be presented to T cells in a variety of ways known in the art. In one common technique, the peptide is “pulsed” or loaded onto antigen presenting cells (“APCs”) such as monocytes or dendritic cells (“DC”) and T cells are contacted with the loaded APCs in vitro or in vivo. Methods of pulsing and loading APCs with peptides and proteins are well known in the art. See, e.g., Schueller et al., Int J Oncol 22(6):1397-402 (2003); Einsele et al., Cytotherapy, 4(1):49-54 (2002); Pospisilova et al., Cancer Immunol Immunother 51(2):72-8 (2002); Zhang et al., Cancer Biother Radiopharm 17(6):601-19 (2002); Olasz et al., Int Immunol. 14 (5):493-502 (2002). Typically, the peptide is introduced by passive pulsing, lipofection, or electroporation. In some embodiments, the peptide is encoded in mRNA introduced into DC by pulsing. See, e.g., Kalady et al., J Surg Res 105(1):17-24 (2002). The “loaded” cells are used to sensitize CD8+ cells, such as tumor infiltrating lymphocytes (“TILs”) from prostate cancer tumors, or peripheral blood lymphocytes (“PBL”). The TILs or PBLs are preferably from the subject. However, they should at least be MHC Class-I restricted to the HLA types the subject possesses. The sensitized cells are then administered to the subject.

Alternatively, hematopoietic stem cells can be transformed with a nucleic acid encoding a peptide of the invention and differentiated into a dendritic cell expressing the peptide on its surface (dendritic cells are known to be APCs). Techniques for introducing nucleic acids into hematopoietic stem cells and differentiating the cells into dendritic cells which express a protein encoded in the nucleic acid are taught in, for example, WO 97/29183. The dendritic cells differentiated from the transformed stem cells were shown to activate T cells against the protein encoded in the nucleic acid with which the stem cells were transformed.

In another method, a nucleic acid sequence encoding one or more peptides of the invention (such as the nucleic acid sequences of SEQ ID NOS:27-31) is administered to the subject. Optionally, the nucleic acid sequence encodes two or more of the peptides. The nucleic acid optionally also can encode cytokines (e.g., IL-2), a costimulatory molecule or other genes that enhance the immune response. In one method of administration, the nucleic acid is introduced directly into superficial layers of the skin or into muscle cells by a jet of compressed gas or the like. Methods for administering immunostimulatory peptides to a mammalian host by the introduction of one or more naked polynucleotides operatively encoding the immunostimulatory peptides are well known and are taught, for example, in U.S. Pat. No. 5,830,877 and International Publication Nos. WO 99/52483 and 94/21797. Devices for accelerating particles into body tissues using compressed gases are described in, for example, U.S. Pat. Nos. 6,592,545, 6,475,181, and 6,328,714. The nucleic acid may be lyophilized and may be complexed, for example, with polysaccharides to form a particle of appropriate size and mass for acceleration into tissue. Conveniently, the nucleic acid can be placed on a gold bead or other particle which provides suitable mass or other characteristics. Use of gold beads to carry nucleic acids into body tissues to induce an immune response is taught in, for example, U.S. Pat. Nos. 4,945,050 and 6,194,389.

The nucleic acid can also be introduced into the body in a virus modified to serve as a vehicle without causing pathogenicity. The virus can be, for example, adenovirus, fowlpox virus or vaccinia virus. Upon infection, the infected cells will express the encoded peptide and express the antigenic determinant on the cell surface in combination with the HLA molecule which binds peptides having the same motif as the antigenic determinant. These cells will then stimulate the activation of CTLs that recognize the presented antigen, resulting in destruction of cancer cells that also bear the determinant.

In another method, recombinant bacteria that express the epitope, such as Bacillus Calmette-Guerin (BCG), Salmonella or Listeria, optionally also encoding cytokines, costimulatory molecules or other genes to enhance the immune response, are administered to the subject.

In another method, cells expressing the antigen are administered to the subject. This includes, for example, dendritic cells pulsed with peptides of the invention, cells transfected with nucleic acids encoding polypeptides of the invention, HLA and B7 genes. The multiple transfection results in the production of several components necessary for presenting the antigenic determinant on the cell surface. In one embodiment, the molecule is a fusion protein in which the polypeptide bearing the antigenic determinant is fused to an HLA molecule (usually through a linker) so as to improve binding of the peptide to the HLA molecule. In one embodiment, the cell is an antigen presenting cell. Preferably, the cells are eukaryotic cells, more preferably, mammalian cells, more preferably, human cells, more preferably autologous human cells derived from the subject.

In a supplemental method, any of these immunotherapies is augmented by administering a cytokine, such as IL-2, IL-3, GM-CSF, or interferons.

In addition to the methods for evaluating immunogenicity of peptides set forth above, immunogenicity can also be evaluated by: evaluation of primary T cell cultures from normal individuals (see, e.g., Wentworth, P. A. et al., Mol. Immunol. 32:603, 1995; Celis, E. et al., Proc. Natl. Acad. Sci. USA 91:2105, 1994; Tsai, V. et al., J. Immunol. 158:1796, 1997; Kawashima, I. et al., Human Immunol. 59:1, 1998); by immunization of HLA transgenic mice (see, e.g., Wentworth, P. A. et al., J. Immunol. 26:97, 1996; Wentworth, P. A. et al., Int. Immunol. 8:651, 1996; Alexander, J. et al., J. Immunol. 159:4753, 1997), and by demonstration of recall T cell responses from patients who have been effectively vaccinated or who have a tumor; (see, e.g., Rehermann, B. et al., J. Exp. Med. 181:1047, 1995; Doolan, D. L. et al., Immunity 7:97, 1997; Bertoni, R. et al., J. Clin. Invest. 100:503, 1997; Threlkeld, S. C. et al., J. Immunol. 159:1648, 1997; Diepolder, H. M. et al., J. Virol. 71:6011, 1997).

In choosing CTL-inducing peptides of interest for vaccine compositions, peptides with higher binding affinity for class I HLA molecules are generally preferable. Peptide binding can be assessed, for example, by testing the ability of a candidate peptide to bind to a purified HLA molecule in vitro, or by the ability of the peptide to stabilize expression of the relevant HLA molecule on a TAB-deficient cell, such as T2, that does not load endogenous peptide.

### Producing Peptides of the Invention

The present invention provides isolated immunogenic peptides of fifty amino acid residues or fewer, comprising the amino acid sequence X₁X₂X₃PSAPSPX₄ (SEQ ID NO:5), wherein X₁ can be any amino acid and is preferably G or Y; X₂ can be selected from the group consisting of L, M, A, I, V; and T, with L and M being preferred; X₃ can be a hydrophobic residue or A; and X₄ may be V, M, L, A, I, or T, and is preferably V, or comprising an amino sequence of SEQ ID NOS:38-40, or both an amino acid sequence of SEQ ID NO:5 and one or more of SEQ ID NOS:38-40. To be considered a peptide of this invention, the peptide must bind to HLA-A2 and, when presented in conjunction with HLA-A2, induce cytotoxic T-cells to lyse cells expressing XAGE-1. These immunogenic peptides may be synthesized by any of the techniques that are known to those skilled in the peptide art, including recombinant DNA techniques.

Synthetic chemistry techniques, such as a solid-phase Merrifield-type synthesis, may be preferred by some practitioners for reasons of purity, antigenic specificity, freedom from undesired side products, ease of production and the like. Summaries of some of the many techniques available can be found in J. M. Steward & J. D. Young, SOLID PHASE PEPTIDE SYNTHESIS, W.H. Freeman Co., San Francisco, (1969); M. Bodanszky et al., PEPTIDE SYNTHESIS, John Wiley & Sons, Second Edition, (1976); and J. Meienhofer, HORMONAL PROTEINS AND PEPTIDES, Vol. 2, p. 46, Academic Press, New York (1983) for solid phase peptide synthesis, and E. Schroder & K. Kubke, 1 THE PEPTIDES, Academic Press, New York (1965) for classical solution synthesis, each being hereby incorporated herein by reference. Appropriate protective groups usable in such synthesis are described in the above texts and in J. F. W. McOmie, PROTECTIVE GROUPS IN ORGANIC CHEMISTRY, Plenum Press, New York (1973), which is also incorporated herein by reference. Simplified methods for solid phase synthesis of peptides on a small scale also are known. See for instance, Houghten, R. A., Proc. Natl. Acad. Sci. U.S.A. 82:5131-5135 (1985); and Houghton, M., Q.-L. Choo, & G. Kuo, European Patent Application 88310922 (1988).

Alternatively, recombinant DNA technology may be employed wherein a nucleotide sequence which encodes an immunogenic peptide of the invention is inserted into an expression vector, transformed or transfected into an appropriate host cell and cultivated under conditions suitable for expression. These procedures are generally known in the art, as described generally in Sambrook et al., Molecular Cloning, A Laboratory Manual, Cold Spring Harbor Press, Cold Spring Harbor, N.Y. (1989), and CURRENT PROTOCOLS IN MOLECULAR BIOLOGY, F. M. Ausubel et al., eds., (Current Protocols, Greene Publishing Associates, Inc. and John Wiley & Sons, Inc.) (“Ausubel”).

Useful promoters for such purposes include a metallothionein promoter, a constitutive adenovirus major late promoter, a dexamethasone-inducible MMTV promoter, a SV40 promoter, a MRP polIII promoter, a constitutive MPSV promoter, a tetracycline-inducible CMV promoter (such as the human immediate-early CMV promoter), and a constitutive CMV promoter. A plasmid useful for gene therapy can comprise other functional elements, such as selectable markers, identification regions, and other genes.

Expression vectors useful in this invention depend on their intended use. Such expression vectors must, of course, contain expression and replication signals compatible with the host cell. Expression vectors useful for expressing bioactive conjugates include viral vectors such as retroviruses, adenoviruses and adeno-associated viruses, plasmid vectors, cosmids, and the like. Viral and plasmid vectors are preferred for transfecting mammalian cells. The expression vector pcDNA3 (Invitrogen, San Diego, Calif.), in which the expression control sequence comprises the CMV promoter, provides good rates of transfection and expression. Adeno-associated viral vectors are useful in the gene therapy methods of this invention.

A variety of means are available for delivering polynucleotides to cells including, for example, direct uptake of the molecule by a cell from solution, facilitated uptake through lipofection (e.g., liposomes or immunoliposomes), particle-mediated transfection, and intracellular expression from an expression cassette having an expression control sequence operably linked to a nucleotide sequence that encodes the inhibitory polynucleotide. See also U.S. Pat. No. 5,272,065 (Inouye et al.); METHODS IN ENZYMOLOGY, vol. 185, Academic Press, Inc., San Diego, Calif. (D. V. Goeddel, ed.) (1990) or M. Krieger, GENE TRANSFER AND EXPRESSION—A LABORATORY MANUAL, Stockton Press, New York, N.Y., (1990). Recombinant DNA expression plasmids can also be used to prepare the polynucleotides of the invention for delivery, although it may be more economical to make short oligonucleotides by in vitro chemical synthesis.

The construct can also contain a tag to simplify isolation of the protein. For example, a polyhistidine tag of, e.g., six histidine residues, can be incorporated at the amino terminal end of the protein. The polyhistidine tag allows convenient isolation of the protein in a single step by nickel-chelate chromatography.

### Preparation of Nucleic Acids Expressing Peptides of the Invention

Nucleic acid sequences encoding peptides of the invention can be prepared by any suitable method including, for example, cloning of appropriate sequences or by direct chemical synthesis by methods such as the phosphotriester method of Narang, et al., Meth. Enzymol. 68:90-99 (1979); the phosphodiester method of Brown, et al., Meth. Enzymol. 68:109-151 (1979); the diethylphosphoramidite method of Beaucage, et al., Tetra. Lett. 22:1859-1862 (1981); the solid phase phosphoramidite triester method described by Beaucage & Caruthers, Tetra. Letts. 22(20):1859-1862 (1981), e.g., using an automated synthesizer as described in, for example, Needham-VanDevanter, et al. Nucl. Acids Res. 12:6159-6168 (1984); and, the solid support method of U.S. Pat. No. 4,458,066. Chemical synthesis produces a single stranded oligonucleotide. This may be converted into double stranded DNA by hybridization with a complementary sequence, or by polymerization with a DNA polymerase using the single strand as a template. One of skill would recognize that while chemical synthesis of DNA is limited to sequences of about 100 bases, longer sequences may be obtained by the ligation of shorter sequences.

In a preferred embodiment, the nucleic acid sequences of this invention are prepared by cloning techniques. Examples of appropriate cloning and sequencing techniques, and instructions sufficient to direct persons of skill through many cloning exercises are found in Sambrook, et al., supra, Berger and Kimmel (eds.), supra, and Ausubel, supra. Product information from manufacturers of biological reagents and experimental equipment also provide useful information. Such manufacturers include the SIGMA chemical company (Saint Louis, Mo.), R&D systems (Minneapolis, Minn.), CLONTECH Laboratories, Inc. (Palo Alto, Calif.), Chem Genes Corp., Aldrich Chemical Company (Milwaukee, Wis.), Glen Research, Inc., Fluka Chemica-Biochemika Analytika (Fluka Chemie AG, Buchs, Switzerland), Invitrogen, San Diego, Calif., and Applied Biosystems (Foster City, Calif.), as well as many other commercial sources known to one of skill.

Nucleic acids encoding peptides of the invention can be modified by site-directed mutagenesis is well known in the art, and can be amplified by in vitro methods. Amplification methods include polymerase chain reaction (PCR), the ligase chain reaction (LCR), the transcription-based amplification system (TAS), and the self-sustained sequence replication system (3SR). A wide variety of cloning methods, host cells, and in vitro amplification methodologies are well known to persons of skill.

In a preferred embodiment, nucleic acids encoding peptides of the invention are prepared by inserting cDNA which encodes the peptide into a vector. The insertion is made so that the peptide and any cytokines or other immunogenic molecule or antagonist to an immunosuppressive molecule to be expressed with it are read in frame, that is in one continuous polypeptide. Once the nucleic acids encoding the peptide are isolated and cloned, one may express the peptide in a recombinantly engineered cell such as bacteria, plant, yeast, insect or mammalian cell. It is expected that those of skill in the art are knowledgeable in the numerous expression systems available for expression of proteins including E. coli, other bacterial hosts, yeast, and various higher eukaryotic cells such as the COS, CHO, HeLa and myeloma cell lines. No attempt to describe in detail the various methods known for the expression of proteins in prokaryotes or eukaryotes will be made.

One of skill will recognize that modifications can be made to a nucleic acid encoding a peptide of the invention without diminishing its biological activity. Some modifications may be made to facilitate the cloning, expression, or incorporation of the targeting molecule into a fusion protein. Such modifications are well known to those of skill in the art and include, for example, termination codons, a methionine added at the amino terminus to provide an initiation site, additional amino acids placed on either terminus to create conveniently located restriction sites, or additional amino acids (such as poly His) to aid in purification steps.

Once expressed, the recombinant peptides can be purified according to standard procedures of the art, including ammonium sulfate precipitation, affinity columns, column chromatography, and the like (see, generally, R. Scopes, PROTEIN PURIFICATION, Springer-Verlag, N.Y. (1982)). Substantially pure compositions of at least about 90 to 95%-homogeneity are preferred, and 98 to 99% or more homogeneity are most preferred for pharmaceutical uses. Once purified, partially or to homogeneity as desired, if to be used therapeutically, the peptides should be substantially free of endotoxin.

### Compositions of Peptides of the Invention in Pharmaceutically Acceptable Carriers

In another aspect, this invention provides compositions that comprise a composition of this invention in a pharmaceutically acceptable carrier and a.

In one group of embodiments, the composition comprises a peptide of the invention in an amount effective to elicit a cell-mediated immune response in a subject. Such pharmaceutical compositions are useful in stimulating or enhancing an immune response to XAGE-1-expressing cancers. These compositions are preferably administered intradermally, subcutaneously, or intramuscularly.

The compositions for administration will commonly comprise a solution of the peptide dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of peptide in these formulations can vary widely, and will be selected primarily based on fluid volumes, viscosities, body weight and the like in accordance with the particular mode of administration selected.

The compositions of the invention are administered to a patient in an amount sufficient to elicit an effective immune response, preferably a CTL response, to cells expressing XAGE-1. An amount adequate to accomplish this is defined as “therapeutically effective dose.”

Amounts effective for this use will depend on, e.g., the peptide and/or protein composition, the manner of administration, the stage and severity of XAGE-1-expressing disease, the weight and general state of health of the patient, and the judgment of the prescribing physician, but generally range for the initial immunization (for therapeutic or prophylactic administration) from about 0.001 to about 200 mg/kg, more preferably about 0.01 to about 100 mg/kg, most preferably about 0.1 to 50 mg/kg peptide. The initial immunization may be followed by boosting dosages of from about 0.001 to about 100 mg/kg, more preferably about 0.01 to about 50 mg/kg peptide pursuant to a boosting regimen over weeks to months, depending upon the patient's response and condition determined by measuring specific CTL activity, in the patient's blood.

Actual methods for preparing administrable compositions will be known or apparent to those skilled in the art and are described in more detail in such publications as REMINGTON'S PHARMACEUTICAL SCIENCE, 19TH ED., Mack Publishing Company, Easton, Pa. (1995).

In another embodiment, the composition comprises a nucleic acid molecule comprising a nucleotide sequence encoding a peptide of the invention, which nucleic acid molecule is in an amount effective to elicit an immune response against RAGE-1-expressing cells in a subject. Such composition also are useful in the therapeutic methods of this invention.

The compositions of this invention can be prepared in unit dosage forms for administration to a subject. The amount and timing of administration are at the discretion of the treating physician to achieve the desired purposes.

The compositions of the present invention can be administered to inhibit the growth of cells of XAGE-1 expressing cancers. In these applications, compositions are administered to a patient suffering from a disease, in an amount sufficient to inhibit growth of XAGE-1-expressing cells. An amount adequate to accomplish this is defined as a “therapeutically effective dose.” Amounts effective for this use will depend upon the severity of the disease and the general state of the patient's health. An effective amount of the compound is that which provides either subjective relief of a symptom(s) or an objectively identifiable improvement as noted by the clinician or other qualified observer.

Single or multiple administrations of the compositions are administered depending on the dosage and frequency as required and tolerated by the patient. In any event, the composition should provide a sufficient quantity of the proteins of this invention to effectively treat the patient. Preferably, the dosage is administered once but may be applied periodically until either a therapeutic result is achieved or until side effects warrant discontinuation of therapy. Generally, the dose is sufficient to treat or ameliorate symptoms or signs of disease without producing unacceptable toxicity to the patient.

Controlled release parenteral formulations of the immunoconjugate compositions of the present invention can be made as implants, oily injections, or as particulate systems. For a broad overview of protein delivery systems see, Banga, A. J., THERAPEUTIC PEPTIDES AND PROTEINS: FORMULATION, PROCESSING, AND DELIVERY SYSTEMS, Technomic Publishing Company, Inc., Lancaster, Pa., (1995) incorporated herein by reference. Particulate systems include microspheres, microparticles, microcapsules, nanocapsules; nanospheres, and nanoparticles. Microcapsules contain the therapeutic protein as a central core. In microspheres the therapeutic is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly. See, e.g., Kreuter, J., COLLOIDAL DRUG DELIVERY SYSTEMS, J. Kreuter, ed., Marcel Dekker, Inc., New York, N.Y., pp. 219-342 (1994); and Tice & Tabibi, TREATISE ON CONTROLLED DRUG DELIVERY, A. Kydonieus, ed., Marcel Dekker, Inc. New York, N.Y., pp. 315-339, (1992) both of which are incorporated herein by reference.

Polymers can be used for ion-controlled release of immunoconjugate compositions of the present invention. Various degradable and nondegradable polymeric matrices for use in controlled drug delivery are known in the art (Langer, R., Accounts Chem. Res. 26:537-542 (1993)). For example, the block copolymer, polaxamer 407 exists as a viscous yet mobile liquid at low temperatures but forms a semisolid gel at body temperature. It has shown to be an effective vehicle for formulation and sustained delivery of recombinant interleukin-2 and urease (Johnston, et al., Pharm. Res. 9:425-434 (1992); and Pec, et al., J. Parent. Sci. Tech. 44(2):58-65 (1990)). Alternatively, hydroxyapatite has been used as a microcarrier for controlled release of proteins (Ijntema, et al., Int. J. Pharm. 112:215-224 (1994)). In yet another aspect, liposomes are used for controlled release as well as drug targeting of the lipid-capsulated drug (Betgeri, et al., LIPOSOME DRUG DELIVERY SYSTEMS, Technomic Publishing Co., Inc., Lancaster, Pa. (1993)). Numerous additional systems for controlled delivery of therapeutic proteins are known. See, e.g., U.S. Pat. Nos. 5,055,303, 5,188,837, 4,235,871, 4,501,728, 4,837,028 4,957,735 and 5,019,369, 5,055,303; 5,514,670; 5,413,797; 5,268,164; 5,004,697; 4,902,505; 5,506,206, 5,271,961; 5,254,342 and 5,534,496, each of which is incorporated herein by reference.

Either peptides of the invention or nucleic acids encoding them, or both, may also be administered via liposomes. Liposomes are useful in increasing the half-life of the peptides and in protecting the nucleic acids from extracellular nucleases. Liposomes include emulsions, foams, micelles, insoluble monolayers, liquid crystals, phospholipid dispersions, lamellar layers and the like. In liposome preparations, the peptide to be delivered may be incorporated as part of a liposome, alone or in conjunction with a molecule that binds to, e.g., a receptor prevalent among lymphoid cells, such as monoclonal antibodies that bind to the CD45 antigen, or with other therapeutic or immunogenic compositions. Thus, liposomes filled with a desired peptide of the invention can be directed to the site of lymphoid cells, where the liposomes then deliver the selected therapeutic/immunogenic peptide compositions.

Among various uses of the immunotoxins of the present invention are included a variety of disease conditions caused by specific human cells that may be eliminated by the toxic action of the fusion protein. One preferred application for the immunotoxins of the invention is the treatment of malignant cells expressing XAGE-1. Exemplary malignant cells include ovarian, lung, prostate, breast, and pancreatic cancers, as well as XAGE-1-expressing muscle and bone cancers.

### EXAMPLES

The following examples are offered to illustrate, but not to limit the claimed invention.

### Example 1

This Example sets forth how binding assays were performed.

Binding affinity to HLA-A2 of synthetic peptides derived from the XAGE-1 amino acid sequence was measured by using T2 cells. The T2 cell line is a T-B fusion hybridoma cell line deficient in TAP1 and TAP2 gene expression available from the ATCC under accession number CRL-1992. T2 cells suspended in Dulbecco's modified Eagles' medium (DMEM) containing 2.5% fetal calf serum (FCS) were incubated overnight with different concentrations of the peptides and 10 μg/ml of β2-microglobulin. The cells were washed twice with phosphate buffered saline (PBS) containing 2% FCS and incubated on ice with anti-HLA-A2 monoclonal antibody (BB7.2, produced by ATCC cell line HB-82) for 30 min. After a single wash, the cells were stained with FITC-labeled goat anti-mouse Ig antibody for 30 min on ice. The cells were washed twice, and expression of HLA-A2 was measured by flow cytometry. The expression level of HLA-A2 was determined as follows: mean fluorescent index (F.I.)=(mean fluorescent intensity of the sample with peptide−mean fluorescent intensity of the sample without peptide)/mean fluorescent intensity of the sample without peptide; mean fluorescent intensity=geometric mean fluorescent intensity of the sample stained with both 1st and 2nd antibody−geometric mean fluorescent intensity of the sample stained with 2nd antibody alone. The results are shown in FIG. 2.

### Example 2

This Example sets forth immunization protocols and CTL assays used in studies underlying the invention.

Xage-1 14 peptide (50 nmol) was emulsified in incomplete Freund's adjuvant with 50 nmol of a helper epitope derived from HCV core128-140, 5 μg of IL-12, and 5 μg of GM-CSF. Transgenic A2 Kb mice were immunized s.c. with the emulsified peptide and given a booster shot at 2 weeks. Two weeks after the boost, the mice were euthanized, and spleens were removed. The spleen cells were stimulated in vitro by xage-1 14 peptide (SEQ ID NO:6)-pulsed (10 μM) immune spleen cells in 10% T-stim-CTM, RPMI 1640 containing 10% FCS, L-glutamine, sodium pyruvate, nonessential amino acids, penicillin, streptomycin and 2-mercaptoethanol. On day 7, the cells were restimulated with xage-1 14 peptide (SEQ ID NO:6)-pulsed naïve A2 Kb spleen cells. A week after the second stimulation, cells were harvested and measured for cytotoxic activity. The cytotoxic activity was measured by 4-hr ⁵¹Cr-release assay. The percentage of specific ⁵¹Cr release was determined as follows: (experimental release−spontaneous release)/(maximum release−spontaneous release)×100. Maximum release was determined from supernatants of cells that were lysed by addition of 5% Triton X-100. Spontaneous release was determined from target cells incubated without added effector cells.

### Example 3

This Example sets forth testing of potentially immunogenic peptides.

To find potential immunogenic peptide derived from XAGE-1, the xage-1 p16 amino acid sequence was searched for potential HLA-A2 binding peptides using a computer program. A known HLA-A2 binding motif was used to determine putative immunogenic peptides. Three such putative peptides were selected: (a) a peptide containing residues 14-23 of xage-1, (b) a peptide containing residues 33-42 of xage-1 (“xage-1 33”, SEQ ID NO:32), and (c) a peptide containing residues 57-66 of xage-1 (“xage-1 57”, SEQ ID NO:33). See, Table 2, below. The peptides were then synthesized for study.

To test their binding affinity to HLA-A2, the peptides were used in T2 binding assay as described in Example 1. Xage-1 14 (SEQ ID NO:6) showed high binding affinity to HLA-A2 (F.I.50=0.7˜0.8 μM). Xage-1 33 and xage-1 57 showed very low binding affinity to HLA-A2 (F.I.50>50 μM). Thus, the testing established that computer predictions of HLA binding could not reliably predict whether or not a peptide would bind HLA-A2.

Since the peptide xage-1 14 (SEQ ID NO:6) showed high binding affinity to HLA-A2, its ability to induce CTL activity in vivo was examined using HLA-A2 transgenic mice. After giving two doses of the peptide mixed with 1L-12 and GM-CSF emulsified in IFA, spleen cells of HLA-A2 transgenic mice were stimulated twice with xage-1 14 (SEQ ID NO:6)-pulsed antigen presenting cells (APCs), and then tested for specific lytic activity against xage-1 14 (SEQ ID NO:6)-pulsed targets (FIG. 3). Spleen cells from HLA-A2-transgenic mice immunized with xage-1 14 (SEQ ID NO:6) lysed target cells pulsed with peptide, but did not lyse target cells not pulsed with peptide. These results indicate that xage-1 14 (SEQ ID NO:6) not only could bind to HLA-A2 but also could induce peptide-specific CTLs in vivo.

Moreover, spleen cells from HLA-A2-transgenic mice immunized with xage-1 14 were tested for their ability to lyse a HLA-A2 positive human Ewing's sarcoma cell line which expresses XAGE-1 mRNA. The transgenic mice were immunized twice with xage-1 14. Following normal protocols for CTL lysing studies, CTLs from mice immunized with the peptide were then cultured in vitro and further stimulated with peptide pulsed irradiated spleen cells from naive transgenic mice as APCs. The CTLs were then tested for their ability to lyse the Ewing's sarcoma cells. The CTLs lysed the human Ewing's sarcoma cells.

These results demonstrate that human RAGE-1 positive cancer cells naturally process and present xage-1 14 on their cell surface. They further demonstrate that CTLs from animals immunized with the xage-1 14 peptide can specifically recognize cells with endogenously processed xage-1 14 and lyse them.

TABLE 2
Amino acid sequence of peptides derived from XAGE-1

| Name | Sequence | SEQ ID NO: | Position* |
|---|---|---|---|
| XAGE-1 14 | GVFPSAPSPV | 6 | (14-23) |
| XAGE-1 33 | ATRVPEVWIL | 32 | (33-42) |
| XAGE-1 57 | HTASPRSPVM | 33 | (57-66) |

*“Position” identifies the positions the residues occupy in xage-1 p16, counting from the first methionine shown in FIG. 1.

### Example 4

This Example sets forth the results of studies on enhancing HLA-A2 binding with enhanced forms of SEQ ID NO:6.

A series of peptides derived from xage-1 14 were discovered with enhanced binding to HLA-A2. The binding affinity was determined by T2 binding assays, as described in the previous Examples, and was found to be as follows:

TABLE 2
HLA-A2 Binding of Enhanced xage-1 14 Peptides

| | | | Binding |
|---|---|---|---|
| | | | Affinity |
| | | SEQ | to HLA-A2 |
| Name | Peptide | ID NO: | (FI₅₀) |
| 1Y xage-1 14 | YVFPSAPSPV | 7 | 2.36 μM |
| 2L xage-1 14 | GLFPSAPSPV | 8 | 1.86 μM |
| 3M xage-1 14 | GVMPSAPSPV | 9 | 0.28 μM |
| 1Y2L xage-1 14 | YLFPSAPSPV | 10 | 3.04 μM |
| 2L3M xage-1 14 | GLMPSAPSPV | 11 | 0.62 μM |

1. An isolated immunogenic peptide of 50 or fewer amino acids comprising an amino acid sequence X₁X₂X₃PSAPSPX₄ (SEQ ID NO:5), wherein:
X₁ can be any amino acid;
X₂ can be L, M, A, I, V, or T;
X₃ can be a hydrophobic residue, methionine or alanine; and
X₄ can be V, M, L, A, I, or T.

2. An immunogenic peptide of claim 1, wherein X₁ is tyrosine (SEQ ID NO:34).

3. An immunogenic peptide of claim 1, wherein X₂ is leucine (SEQ ID NO:35).

4. An immunogenic peptide of claim 1, wherein X₃ is methionine (SEQ ID NO:36).

5. An immunogenic peptide of claim 1, wherein X₄ is valine (SEQ ID NO:37).

6. An immunogenic peptide of claim 1, which peptide comprises an amino acid sequence selected from the group consisting of GVFPSAPSPV (SEQ ID NO:6), YVFPSAPSPV (SEQ ID NO:7), GLFPSAPSPV (SEQ ID NO:8), GVMPSAPSPV (SEQ ID NO:9), YLFPSAPSPV (SEQ ID NO:10), and GLMPSAPSPV (SEQ ID NO:11).

7. An immunogenic peptide of claim 1, which peptide is a ten amino acid peptide having an amino acid sequence selected from the group consisting of GVFPSAPSPV (SEQ ID NO:6), YVFPSAPSPV (SEQ ID NO:7), GLFPSAPSPV (SEQ ID NO:8), GVMPSAPSPV (SEQ ID NO:9), YLFPSAPSPV (SEQ ID NO:10), and GLMPSAPSPV (SEQ ID NO:11).

8. A composition comprising:
i) an isolated immunogenic peptide of fifty or fewer amino acids comprising the sequence of X₁X₂X₃PSAPSPX₄ (SEQ ID NO:5), wherein:
X₁ can be any amino acid;
X₂ can be L, M, A, I, V, or T;
X₃ can be a hydrophobic residue, methionine, or alanine; and
X₄ can be V, M, L, A, I, or T; and,
ii) a pharmaceutically acceptable carrier.

9. A composition of claim 8, wherein X₁ is tyrosine (SEQ ID NO:34).

10. A composition of claim 8, wherein X₂ is leucine (SEQ ID NO:35).

11. A composition of claim 8, wherein X₃ is methionine (SEQ ID NO:36).

12. A composition of claim 8, wherein X₄ is valine (SEQ ID NO:37).

13. A composition of claim 8, wherein said peptide comprises an amino acid sequence selected from the group consisting of GVFPSAPSPV (SEQ ID NO:6), YVFPSAPSPV (SEQ ID NO:7), GLFPSAPSPV (SEQ ID NO:8), GVMPSAPSPV (SEQ ID NO:9), YLFPSAPSPV (SEQ ID NO:10), and GLMPSAPSPV (SEQ ID NO:11).

14. A composition of claim 8, wherein said peptide is a ten amino acid peptide having an amino acid sequence selected from the group consisting of GVFPSAPSPV (SEQ ID NO:6), YVFPSAPSPV (SEQ ID NO:7), GLFPSAPSPV (SEQ ID NO:8), GVMPSAPSPV (SEQ ID NO:9), YLFPSAPSPV (SEQ ID NO:10), and GLMPSAPSPV (SEQ ID NO:11).

15-20. (canceled)

21. A method of inhibiting growth of an XAGE-1-expressing cancer cell in a subject, said method comprising administering to said subject a purified peptide of fifty or fewer amino acids, said peptide comprising a sequence of X₁X₂X₃PSAPSPX₄ (SEQ ID NO:5), wherein:
X₁ can be any amino acid;
X₂ can be L, M, A, I, V, or T;
X₃ can be a hydrophobic residue, methionine, or alanine; and
X₄ can be V, M, L, A, I, or T
wherein administration of said peptide to said subject stimulates or activates cytotoxic T lymphocytes, thereby inhibiting growth of said XAGE-1-expressing cancer cell.

22. A method of claim 21, wherein X₁ is a tyrosine (SEQ ID NO:34).

23. A method of claim 21, wherein X₂ is a leucine (SEQ ID NO:35).

24. A method of claim 21, wherein X₃ is a methionine (SEQ ID NO:36).

25. A method of claim 21, wherein said peptide comprises an amino acid sequence selected from the group consisting of GVFPSAPSPV (SEQ ID NO:6), YVFPSAPSPV (SEQ ID NO:7), GLFPSAPSPV (SEQ ID NO:8), GVMPSAPSPV (SEQ ID NO:9), YLFPSAPSPV (SEQ ID NO:10), and GLMPSAPSPV (SEQ ID NO:11).

26. A method of claim 21, wherein said peptide has an amino acid sequence selected from the group consisting of GVFPSAPSPV (SEQ ID NO:6), YVFPSAPSPV (SEQ ID NO:7), GLFPSAPSPV (SEQ ID NO:8), GVMPSAPSPV (SEQ ID NO:9), YLFPSAPSPV (SEQ ID NO:10), and GLMPSAPSPV (SEQ ID NO:11).

27. A method of claim 21, further comprising administering an immunostimulant or an antagonist of immunosuppressive cytokines.

28. An isolated nucleic acid encoding a peptide of fifty or fewer amino acids, said peptide comprising a sequence X₁X₂X₃PSAPSPX₄ (SEQ ID NO:5), wherein:
X₁ can be any amino acid;
X₂ can be L, M, A, I, V, or T;
X₃ can be a hydrophobic residue, methionine, or alanine; and
X₄ can be V, M, L, A, I, or T.

29. An isolated nucleic acid of claim 28, wherein X₁ is tyrosine (SEQ ID NO:34).

30. An isolated nucleic acid of claim 28, wherein X₂ is leucine (SEQ ID NO:35).

31. An isolated nucleic acid of claim 28, wherein X₃ is methionine (SEQ ID NO:36).

32. An isolated nucleic acid of claim 28, wherein said peptide comprises an amino acid sequence selected from the group consisting of GVFPSAPSPV (SEQ ID NO:6), YVFPSAPSPV (SEQ ID NO:7), GLFPSAPSPV (SEQ ID NO:8), GVMPSAPSPV (SEQ ID NO:9), YLFPSAPSPV (SEQ ID NO:10), and GLMPSAPSPV (SEQ ID NO:11).

33. An isolated nucleic acid of claim 28, wherein said peptide has an amino acid sequence selected from the group consisting of GVFPSAPSPV (SEQ ID NO:6), YVFPSAPSPV (SEQ ID NO:7), GLFPSAPSPV (SEQ ID NO:8), GVMPSAPSPV (SEQ ID NO:9), YLFPSAPSPV (SEQ ID NO:10), and GLMPSAPSPV (SEQ ID NO:11).

34. A vector comprising a nucleic acid sequence of claim 28 operably linked to a promoter.

35. A vector of claim 34, wherein said nucleic acid sequence encodes a peptide comprising an amino acid sequence selected from the group consisting of GVFPSAPSPV (SEQ ID NO:6), YVFPSAPSPV (SEQ ID NO:7), GLFPSAPSPV (SEQ ID NO:8), GVMPSAPSPV (SEQ ID NO:9), YLFPSAPSPV (SEQ ID NO:10), and GLMPSAPSPV (SEQ ID NO:11).

36. A composition comprising a vector of claim 34 and a pharmaceutically acceptable carrier.

37. A composition of claim 36, wherein said vector encodes a peptide comprising an amino acid sequence selected from the group consisting of GVFPSAPSPV (SEQ ID NO:6), YVFPSAPSPV (SEQ ID NO:7), GLFPSAPSPV (SEQ ID NO:8), GVMPSAPSPV (SEQ ID NO:9), YLFPSAPSPV (SEQ ID NO:10), and GLMPSAPSPV (SEQ ID NO:11).

38-39. (canceled)

40. A method of inhibiting the growth of an XAGE-1-expressing cancer cell in a subject, said method comprising administering to said subject an isolated nucleic acid sequence encoding a peptide of fifty or fewer amino acids, said peptide comprising of the sequence X₁X₂X₃PSAPSPX₄ (SEQ ID NO:5), wherein: X₁ can be any amino acid; X₂ can be L, M, A, I, V, or T; X₃ can be a hydrophobic residue, methionine, or alanine; and X₄ can be V, M, L, A, I, or T; wherein administration of said nucleic acid sequence results in expression of said peptide, which expression stimulates or activates cytotoxic T lymphocytes, thereby inhibiting the growth of said XAGE-1-expressing cancer cell.

41. A method of claim 40 wherein X₁ is tyrosine (SEQ ID NO:34).

42. A method of claim 40 wherein X₂ is leucine (SEQ ID NO:35).

43. A method of claim 40 wherein X₃ is methionine (SEQ ID NO:36).

44. A method of claim 40, wherein said peptide comprises an amino acid sequence selected from the group consisting of GVFPSAPSPV (SEQ ID NO:6), YVFPSAPSPV (SEQ ID NO:7), GLFPSAPSPV (SEQ ID NO:8), GVMPSAPSPV (SEQ ID NO:9), YLFPSAPSPV (SEQ ID NO:10), and GLMPSAPSPV (SEQ ID NO:11).

45. A method of claim 40, wherein said peptide has an amino acid sequence selected from the group consisting of GVFPSAPSPV (SEQ ID NO:6), YVFPSAPSPV (SEQ ID NO:7), GLFPSAPSPV (SEQ ID NO:8), GVMPSAPSPV (SEQ ID NO:9), YLFPSAPSPV (SEQ ID NO:10), and GLMPSAPSPV (SEQ ID NO:11).

46. A method for stimulating or expanding T cells, or both, comprising contacting T cells with a synthetic or recombinant amino acid sequence X₁X₂X₃PSAPSPX₄ (SEQ ID NO:5), wherein: X₁ can be any amino acid; X₂ can be L, M, A, I, V, or T; X₃ can be a hydrophobic residue, methionine, or alanine; and X₄ can be V, M, L, A, I, or T; thereby stimulating or expanding said T cells, or both.

47. A method of claim 46, wherein X₁ is tyrosine (SEQ ID NO:34).

48. A method of claim 46, wherein X₂ is leucine (SEQ ID NO:35).

49. A method of claim 46, wherein X₃ is methionine (SEQ ID NO:36).

50. A method of claim 46, wherein said peptide comprises an amino acid sequence selected from the group consisting of GVFPSAPSPV (SEQ ID NO:6), YVFPSAPSPV (SEQ ID NO:7), GLFPSAPSPV (SEQ ID NO:8), GVMPSAPSPV (SEQ ID NO:9), YLFPSAPSPV (SEQ ID NO:10), and GLMPSAPSPV (SEQ ID NO:11).

51. A method of claim 46, wherein said peptide has an amino acid sequence selected from the group consisting of GVFPSAPSPV (SEQ ID NO:6), YVFPSAPSPV (SEQ ID NO:7), GLFPSAPSPV (SEQ ID NO:8), GVMPSAPSPV (SEQ ID NO:9), YLFPSAPSPV (SEQ ID NO:10), and GLMPSAPSPV (SEQ ID NO:11).

52. A method of claim 46, wherein said T cells are isolated from bone marrow, or a fraction thereof, of a patient.

53. A method of claim 46, wherein said T cells are isolated from peripheral blood, or a fraction thereof, of a patient.

54. A method of claim 46, wherein said T cells are contacted with said peptide by contacting said T cells with an antigen presenting cell pulsed with, transduced to express, or differentiated from a cell transduced with a nucleic acid encoding, said peptide.

55. A method of claim 46, wherein said T cells are contacted with an antigen presenting cell pulsed with, transduced to express, or differentiated from a cell transduced with a nucleic acid encoding, a peptide having an amino acid sequence selected from the group consisting of GVFPSAPSPV (SEQ ID NO:6), YVFPSAPSPV (SEQ ID NO:7), GLFPSAPSPV (SEQ ID NO:8), GVMPSAPSPV (SEQ ID NO:9), YLFPSAPSPV (SEQ ID NO:10), and GLMPSAPSPV (SEQ ID NO:11).

56. A method of claim 46, wherein said T cells are CD8+ T cells.

57. A method for stimulating or expanding T cells comprising contacting said T cells with an antigen presenting cell pulsed with, transduced to express, or differentiated from a cell transduced with a nucleic acid encoding, an amino acid sequence of X₁X₂X₃PSAPSPX₄ (SEQ ID NO:5), wherein: X₁ can be any amino acid; X₂ can be L, M, A, I, V, or T; X₃ can be a hydrophobic residue, methionine, or alanine; and X₄ can be V, M, L, A, I, or T.

58. A method of claim 57, wherein X₁ is tyrosine (SEQ ID NO:34).

59. A method of claim 57, wherein X₂ is leucine (SEQ ID NO:35).

60. A method of claim 57, wherein X₃ is alanine (SEQ ID NO:36).

61. A method of claim 57, wherein said peptide comprises an amino acid sequence selected from the group consisting of GVFPSAPSPV (SEQ ID NO:6), YVFPSAPSPV (SEQ ID NO:7), GLFPSAPSPV (SEQ ID NO:8), GVMPSAPSPV (SEQ ID NO:9), YLFPSAPSPV (SEQ ID NO:10), and GLMPSAPSPV (SEQ ID NO:11).

62. A method of claim 57, wherein said peptide has an amino acid sequence selected from the group consisting of GVFPSAPSPV (SEQ ID NO:6), YVFPSAPSPV (SEQ ID NO:7), GLFPSAPSPV (SEQ ID NO:8), GVMPSAPSPV (SEQ ID NO:9), YLFPSAPSPV (SEQ ID NO:10), and GLMPSAPSPV (SEQ ID NO:11).

63. A method of inhibiting the growth of a cancer cell expressing XAGE-1 comprising contacting said cell with an isolated cytotoxic T lymphocyte specific for a peptide comprising an amino acid sequence of X₁X₂X₃PSAPSPX₄ (SEQ ID NO:5), wherein: X₁ can be any amino acid; X₂ can be L, M, A, I, V, or T; X₃ can be a hydrophobic residue, methionine, or alanine; and X₄ can be V, M, L, A, I, or T.

64. A method of claim 63, wherein said peptide comprises an amino acid sequence selected from the group consisting of GVFPSAPSPV (SEQ ID NO:6), YVFPSAPSPV (SEQ ID NO:7), GLFPSAPSPV (SEQ ID NO:8), GVMPSAPSPV (SEQ ID NO:9), YLFPSAPSPV (SEQ ID NO:10), and GLMPSAPSPV (SEQ ID NO:11).

65. A method of claim 63, wherein said peptide has an amino acid sequence selected from the group consisting of GVFPSAPSPV (SEQ ID NO:6), YVFPSAPSPV (SEQ ID NO:7), GLFPSAPSPV (SEQ ID NO:8), GVMPSAPSPV (SEQ ID NO:9), YLFPSAPSPV (SEQ ID NO:10), and GLMPSAPSPV (SEQ ID NO:11).

66-74. (canceled)

75. A peptide of claim 1, wherein said peptide is 20 amino acids or fewer.

76. A composition of claim 8, wherein said peptide is 20 amino acids or fewer.

77. A method of claim 21, wherein said peptide is 20 amino acids or fewer.

78. A nucleic acid of claim 28, wherein said peptide is 20 amino acids or fewer.

79. A method of claim 40, wherein said peptide is 20 amino acids or fewer.


SEQUENCE LISTING (45 sequences)

SEQ ID NO: 1 - DNA, 246 bp, Homo sapiens
  xage-1 p9, 9kD protein expressed from XAGE-1 gene
atggagagccccaaaaagaagaaccagcagctgaaagtcgggatcctacacctgggcagcagacagaagaagatcaggatacagctgagatcccagtgcgcgacatggaaggtgatctgcaagagctgcatcagtcaaacaccggggataaatctggatttgggttccggcgtcaaggtgaagataatacctaaagaggaacactgtaaaatgccagaagcaggtgaagagcaaccacaagtttaa

SEQ ID NO: 2 - protein, 81 aa, Homo sapiens
  xage-1 p9
MESPKKKNQQLKVGILHLGSRQKKIRIQLRSQCATWKVICKSCISQTPGINLDLGSGVKVKIIPKEEHCKMPEAGEEQPQV

SEQ ID NO: 3 - DNA, 441 bp, Homo sapiens
  xage-1 p16, 16.3 kD protein expressed from XAGE-1 gene
atgctcctttggtgcccacctcagtgcgcatgttcactgggcgtcttcccatcggccccttcgccagtgtggggaacgcggcggagctgtgagccggcgactcgggtccctgaggtctggattctttctccgctactgagacacggcggacacacacaaacacagaaccacacagccagtcccaggagcccagtaatggagagccccaaaaagaagaaccagcagctgaaagtcgggatcctacacctgggcagcagacagaagaagatcaggatacagctgagatcccagtgcgcgacatggaaggtgatctgcaagagctgcatcagtcaaacaccggggataaatctggatttgggttccggcgtcaaggtgaagataatacctaaagaggaacactgtaaaatgccagaagcaggtgaagagcaaccacaagtttaa

SEQ ID NO: 4 - protein, 146 aa, Homo sapiens
  xage-1 p16
MLLWCPPQCACSLGVFPSAPSPVWGTRRSCEPATRVPEVWILSPLLRHGGHTQTQNHTASPRSPVMESPKKKNQQLKVGILHLGSRQKKIRIQLRSQCATWKVICKSCISQTPGINLDLGSGVKVKIIPKEEHCKMPEAGEEQPQV

SEQ ID NO: 5 - protein, 10 aa, Artificial Sequence
  Description of Artificial Sequenceimmunogenic peptide derived from xage-1 14
XXXPSAPSPX

SEQ ID NO: 6 - protein, 10 aa, Artificial Sequence
  Description of Artificial Sequencexage-1 14, immunogenic amino terminal end of xage-1, xage-1 residues 14-23
GVFPSAPSPV

SEQ ID NO: 7 - protein, 10 aa, Artificial Sequence
  Description of Artificial Sequence1Y xage-1 14, variant of xage-1 14, immunogenic peptide derived from xage-1 14
YVFPSAPSPV

SEQ ID NO: 8 - protein, 10 aa, Artificial Sequence
  Description of Artificial Sequence2L xage-1 14, variant of xage-1 14, immunogenic peptide derived from xage-1 14
GLFPSAPSPV

SEQ ID NO: 9 - protein, 10 aa, Artificial Sequence
  Description of Artificial Sequence3M xage-1 14, variant of xage-1 14, immunogenic peptide derived from xage-1 14
GVMPSAPSPV

SEQ ID NO: 10 - protein, 10 aa, Artificial Sequence
  Description of Artificial Sequence1Y2L xage-1 14, variant of xage-1 14, immunogenic peptide derived from xage-1 14
YLFPSAPSPV

SEQ ID NO: 11 - protein, 10 aa, Artificial Sequence
  Description of Artificial Sequence2L3M xage-1 14, variant of xage-1 14, immunogenic peptide derived from xage-1 14
GLMPSAPSPV

SEQ ID NO: 12 - protein, 10 aa, Artificial Sequence
  Description of Artificial Sequencemodified xage-1 14 peptide, immunogenic peptide derived from xage-1 14
GVWPSAPSPV

SEQ ID NO: 13 - protein, 10 aa, Artificial Sequence
  Description of Artificial Sequencemodified xage-1 14 peptide, immunogenic peptide derived from xage-1 14
GVYPSAPSPV

SEQ ID NO: 14 - protein, 10 aa, Artificial Sequence
  Description of Artificial Sequencemodified xage-1 14 peptide, immunogenic peptide derived from xage-1 14
TVWPSAPSPM

SEQ ID NO: 15 - protein, 10 aa, Artificial Sequence
  Description of Artificial Sequencemodified xage-1 14 peptide, immunogenic peptide derived from xage-1 14
SMYPSAPSPI

SEQ ID NO: 16 - protein, 10 aa, Artificial Sequence
  Description of Artificial Sequencemodified xage-1 14 peptide, immunogenic peptide derived from xage-1 14
SVFPSAPSPT

SEQ ID NO: 17 - protein, 10 aa, Artificial Sequence
  Description of Artificial Sequencemodified xage-1 14 peptide, immunogenic peptide derived from xage-1 14
GVWPSAPSPM

SEQ ID NO: 18 - protein, 10 aa, Artificial Sequence
  Description of Artificial Sequencemodified xage-1 14 peptide, immunogenic peptide derived from xage-1 14
SVWPSAPSPV

SEQ ID NO: 19 - protein, 10 aa, Artificial Sequence
  Description of Artificial Sequencemodified xage-1 14 peptide, immunogenic peptide derived from xage-1 14
GLWPSAPSPV

SEQ ID NO: 20 - protein, 10 aa, Artificial Sequence
  Description of Artificial Sequencemodified xage-1 14 peptide, immunogenic peptide derived from xage-1 14
IVWPSAPSPV

SEQ ID NO: 21 - protein, 10 aa, Artificial Sequence
  Description of Artificial Sequencemodified xage-1 14 peptide, immunogenic peptide derived from xage-1 14
GLAPSAPSPV

SEQ ID NO: 22 - protein, 10 aa, Artificial Sequence
  Description of Artificial Sequencemodified xage-1 14 peptide, immunogenic peptide derived from xage-1 14
GVAPSAPSPV

SEQ ID NO: 23 - protein, 10 aa, Artificial Sequence
  Description of Artificial Sequencemodified xage-1 14 peptide, immunogenic peptide derived from xage-1 14
YLFPSAPSPM

SEQ ID NO: 24 - protein, 10 aa, Artificial Sequence
  Description of Artificial Sequencemodified xage-1 14 peptide, immunogenic peptide derived from xage-1 14
YLAPSAPSPI

SEQ ID NO: 25 - protein, 10 aa, Artificial Sequence
  Description of Artificial Sequencemodified xage-1 14 peptide, immunogenic peptide derived from xage-1 14
YLAPSAPSPV

SEQ ID NO: 26 - DNA, 30 bp, Artificial Sequence
  Description of Artificial Sequencenucleic acid sequence encoding SEQ ID NO6 native sequence
ggcgtcttcccatcggccccttcgccagtg

SEQ ID NO: 27 - DNA, 30 bp, Artificial Sequence
  Description of Artificial Sequencenucleic acid sequence encoding SEQ ID NO9 preferred form
ggcgtcatgccatcggccccttcgccagtg

SEQ ID NO: 28 - DNA, 30 bp, Artificial Sequence
  Description of Artificial Sequencenucleic acid sequence encoding SEQ ID NO11 preferred form
ggccttatgccatcggccccttcgccagtg

SEQ ID NO: 29 - DNA, 30 bp, Artificial Sequence
  Description of Artificial Sequencenucleic acid sequence encoding SEQ ID NO11 preferred form
ggcctcatgccatcggccccttcgccagtg

SEQ ID NO: 30 - DNA, 30 bp, Artificial Sequence
  Description of Artificial Sequencenucleic acid sequence encoding SEQ ID NO11 preferred form
ggcctaatgccatcggccccttcgccagtg

SEQ ID NO: 31 - DNA, 30 bp, Artificial Sequence
  Description of Artificial Sequencenucleic acid sequence encoding SEQ ID NO11 preferred form
ggcctgatgccatcggccccttcgccagtg

SEQ ID NO: 32 - protein, 10 aa, Artificial Sequence
  Description of Artificial Sequencexage-1 33, residues 33-42 of xage-1
ATRVPEVWIL

SEQ ID NO: 33 - protein, 10 aa, Artificial Sequence
  Description of Artificial Sequencexage-1 57, residues 57-66 of xage-1
HTASPRSPVM

SEQ ID NO: 34 - protein, 10 aa, Artificial Sequence
  Description of Artificial Sequenceimmunogenic peptide derived from xage-1 14 where X-1 is Tyr
YXXPSAPSPX

SEQ ID NO: 35 - protein, 10 aa, Artificial Sequence
  Description of Artificial Sequenceimmunogenic peptide derived from xage-1 14 where X-2 is Leu
XLXPSAPSPX

SEQ ID NO: 36 - protein, 10 aa, Artificial Sequence
  Description of Artificial Sequenceimmunogenic peptide derived from xage-1 14 where X-3 is Met
XXMPSAPSPX

SEQ ID NO: 37 - protein, 10 aa, Artificial Sequence
  Description of Artificial Sequenceimmunogenic peptide derived from xage-1 14 where X-4 is Val
XXXPSAPSPV

SEQ ID NO: 38 - protein, 9 aa, Artificial Sequence
  Description of Artificial Sequence9-mer created from SEQ ID NO5 by omitting Pro at position 9
XXXPSAPSX

SEQ ID NO: 39 - protein, 9 aa, Artificial Sequence
  Description of Artificial Sequence9-mer created from SEQ ID NO5 by omitting Ser at position 8
XXXPSAPPX

SEQ ID NO: 40 - protein, 9 aa, Artificial Sequence
  Description of Artificial Sequence9-mer created from SEQ ID NO5 by omitting Pro at position 7
XXXPSASPX

SEQ ID NO: 41 - protein, 10 aa, Artificial Sequence
  Description of Artificial Sequenceoverall formula for 9-mers created from SEQ ID NO5
XXXPSAXXXX

SEQ ID NO: 42 - DNA, 27 bp, Artificial Sequence
  Description of Artificial Sequenceexemplar nucleic acid encoding a peptide of SEQ ID NO39
ggcgtcttcccatcggccccttcggtg

SEQ ID NO: 43 - DNA, 27 bp, Artificial Sequence
  Description of Artificial Sequenceexemplar nucleic acid encoding a peptide of SEQ ID NO38
ggcgtcttcccatcggcccctccagtg

SEQ ID NO: 44 - DNA, 27 bp, Artificial Sequence
  Description of Artificial Sequenceexemplar nucleic acid encoding a peptide of SEQ ID NO40
ggcgtcttcccatcggcctcgccagtg

SEQ ID NO: 45 - DNA, 637 bp, Homo sapiens
  complete nucleic acid sequence of XAGE-1 with untranslated 5′ and 3′ ends
gtcgttaatggggacctgggaaggagcataggacagggcaaggcgggataaggaggggcaccacagcccttaaggcacgagggaacctcactgcgcatgctcctttggtgcccacctcagtgcgcatgttcactgggcgtcttcccatcggccccttcgccagtgtggggaacgcggcggagctgtgagccggcgactcgggtccctgaggtctggattctttctccgctactgagacacggcggacacacacaaacacagaaccacacagccagtcccaggagcccagtaatggagagccccaaaaagaagaaccagcagctgaaagtcgggatcctacacctgggcagcagacagaagaagatcaggatacagctgagatcccagtgcgcgacatggaaggtgatctgcaagagctgcatcagtcaaacaccggggataaatctggatttgggttccggcgtcaaggtgaagataatacctaaagaggaacactgtaaaatgccagaagcaggtgaagagcaaccacaagtttaaatgaagacaagctgaaacaacgcaagctggttttatattagatatttgacttaaactatctcaataaagttttgcagctttcaccaaaaaaaaaaaaaaa